SEQUENCE LISTING (9 sequences)

SEQ ID NO: 1 - DNA, 1122 bp, Betula pendula
atgggaatcgaaccagagagcttgaagcggttcagtggagcgtggggcgtggcggctaagccctgcgactcctgcaaatcggccgcagcagcggttttctgccgggccgactcggcgtttctgtgcctcacctgcgacgccaaaatccactgcgccaacaagctggcgtcgcggcacgagcgcgtgtggatgtgcgaggtgtgcgagcaggccccagcggccgtcacgtgcaaggccgacgccgccgccctctgtgtcacctgcgacgccgacatccactcggctaacccggtggcgcgtcgccacgagcgcgtccccgtcgagcccttcttcgactctgccgagtccataatcaaatcctcggcgctcagtaatttcctggtggttcccgcggactacaactccaacgacaccaaaaacaaccaccaccatcacgatgaggccgaagcggcgtcgtggattatccccaacccgaatttcagctccaagctcatggatggtccggatttgaaacccggtgaaatgctcttctctgatgtggatccgtttcttgatttcgattatccgaatccgtttcacagtgctggcactgatagcgtggtaccggttcagactaagcccgcctctgctccaatcatcaaccgctccgcagataattgttttgatatcgatttctgtagatccaagctctccgcattcagctatccaacccagtctattagccaaagcatttcgtcgtccgatgttggggtggttccggacgggaattccatgtccgatatatcgtatcctttcggccgcaacatgagtgccgatccgaacgtaccggtctcggggattacaaaccaagtgccgactcagttatgtgggaccgatcgcgaagctagggttttaaggtacagggagaagaggaagaaccggaagttcgagaagactattcgctacgcttcgagaaaagcctacgccgaaacccggccgagaatcaaaggccggttcgcgaaacgcacggagattgactcggaggtggactgcctttacgactcggtgtcagctgcggctttcatatccgacgctcagtacggcgtcgttcctaccttttaaccgtccaagcctttttttctg

SEQ ID NO: 2 - protein, 366 aa, Betula pendula
MGIEPESLKRFSGAWGVAAKPCDSCKSAAAAVFCRADSAFLCLTCDAKIHCANKLASRHERVWMCEVCEQAPAAVTCKADAAALCVTCDADIHSANPVARRHERVPVEPFFDSAESIIKSSALSNFLVVPADYNSNDTKNNHHHHDEAEAASWIIPNPNFSSKLMDGPDLKPGEMLFSDVDPFLDFDYPNPFHSAGTDSVVPVQTKPASAPIINRSADNCFDIDFCRSKLSAFSYPTQSISQSISSSDVGVVPDGNSMSDISYPFGRNMSADPNVPVSGITNQVPTQLCGTDREARVLRYREKRKNRKFEKTIRYASRKAYAETRPRIKGRFAKRTEIDSEVDCLYDSVSAAAFISDAQYGVVPTF

SEQ ID NO: 3 - protein, 220 aa, Betula pendula
NNQSNGFLFGGEVDEYLDLVEYNSCPDQNQFNDQYSRQQQQQQQQLYSVPQKSYGGDSVVPVQCGEAKAQLHHHQQQDFQLGLDFESPKAAYSYHGSISHSVSVSSMDVGVVPESTMSDISISHSRPPKGTIDLFSGPPLQMPAQLTPLDREARVLRYREKKKTRKFEKTIRYASRKAYAETRPRIKGRFAKRTDVEVEVDQMFSTALMAETGYGVVPSF

SEQ ID NO: 4 - protein, 331 aa, Betula pendula
MASKLCDSCKSATATLFCRADSAFLCVSCDSKVHAANKLASRHARVSVCEVCEQAPAHVTCKADAAALCVTCDRDIHSANPLARRHERVPVTPFYDHVAVKSGATANLLDDRYLSDGDVSREEAEAASWLLPNPPNHPANNKVTESPDLNTGQFVFPDMDTYLDLDYGHVDPKMEAQEQNSSGTDGVVPVQSKGVHQLSDHCFDLDFSASKPYVYGYNTHCLSQSVSSSSLDVGVVPDGSAVTDVSDSYAKPVMESANRTVQISAADRVARVLRYREKRKNRKFEKTIRYASRKAYAETRPRIKGRFAKRAEMVEVEVDRVCGGYGVVPSF

SEQ ID NO: 5 - protein, 422 aa, Betula pendula
MICDKNLANAVGGKTARACDSCIRKRARWYCAADDAFLCEACDSSVHSANPLARRHGRVRLKTASFKSSDGDTAVPSWHRGFTRKARTPRHGKPLNHQTPKSDEDQSVRNPFPLVPEVGADETTSHEEEKEEKLLYRVPIFDPFVAELCTTATSKDEAVAANISANRSESCHVDNSLHGCLPSDMDLAEFAADVESLLGRGFENECFGMEGLGLMDCKLEEDADCLGSGRVKLEEEDGMEAVTAACRVDDTEIDMMREPFEFSFDYDSPATCEEEDEKVAVKNGGGLAIAADDAKKKRKNKKILLNLDYEAVIAAWATQGSPWASGDRPNFDPDECWPDCLGIGLHHPYGEFSGSGGNPAMGDGGREARVSRYREKRRTRLFSKKIRYEVRKLNAEKRPRMKGRFVKRTSFAGGPAFPLLTK

SEQ ID NO: 6 - protein, 388 aa, Betula pendula
MIAEKKAANAMGGKTARACDSCLRKRARWFCAADDAFLCQSCDAFVHSANQLASRHERVRLQTSSFKLSTSTVDSSPPPVLPAWHRGFTRKARTPRHYKKEERVFFNPLPFVPEIGSEEASPDAGNDDQLLCRVPVFDPFAAELCNEMGNAIVDERKAAGADDDELDDLHGLLLPSDMELAEFAADVESLLGKGLLDCKEEDDVDGADQGRVKVEEEEELEAIVACQLNPQLDMMRETLDWNFDYYESAAAGEEVERKVVPMAETEMMNIGRECKEEMKREMFLRLNYEAVITAWPGQCSPWTTGTRPQLHPDDCWSDFMGSFPRDVHHHAYGDAGVGSDHVGREARVSRYREKRRTRLFSKKIRYEVRKLNAEKRPRMKGRFVKRTS

SEQ ID NO: 7 - protein, 401 aa, Betula pendula
MIDSSSSSSSKTQNPYKTKQEMTSSPKHAQEDQTISPQKRLCDYCSNATALLYCRADSAKLCFLCDRVIHSGNQLFSKHARSPLCDACDSSPASVFCSTESSVLCQNCDWERHRLSLSPVHNRRPLEGFTGCPSVSELQAIVGFEEVGHKALFLSDESCGDEIDGFSDLFVWDPPFVVGLDDLIGSTDSAHDFEAMRVPPLPKNRNAAACGQYKEEILNQLRHLMKLEPNLNCENVDAEPLASFQSLMPQQNEQPEHMSIGFADNAEPIAFPAFKANAIQWCTDRDEAANQLCLRNSLKKKYLEENPVVPDKRSDTGGSVSHANDGIEAQSLYDPTKYEASSALQRVAPHDLNSQVRDSAISRYKEKRKTRRYDKQIRYESRKVRAESRTRIKGRFAKILH

SEQ ID NO: 8 - DNA, 21 bp, Artificial
  primer
ctctgccgagtccataatcaa

SEQ ID NO: 9 - DNA, 21 bp, Artificial
  primer
taactgagtcggcacttggtt

(19) United States
(12) Patent Application Publication
(10) Pub. No.: US 2007/0011779 A1
(43) Pub. Date: Jan. 11, 2007

(54) Regulation of growth in plants

(76) Inventors: Tapio Palva, Porvoo (FI); Markku Aalto, Sipoo (FI); Pekka Heino, Helsinki (FI); Janne Laine, Mantsala (FI)

Correspondence Address: Leena H Karttunen;Nixon Peabody, 100 Summer Street, Boston, MA, 02110-2131, US

(21) Appl. No.: 10/558,530

(22) PCT Filed: May 26, 2004
(86) PCT No.: PCT/FI04/00324

§ 371 Date: Jan. 20, 2006

(30) Foreign Application Priority Data
May 26, 2003 (FI) 20030792

(51) Int. Cl. A01H 1/00; C07H 21/04; C12N 15/82; C12N 5/04

(52) U.S. Cl. 800/287; 435/419; 435/468; 536/23.6

(57) ABSTRACT

The present invention is related to a birch BpCol2 nucleic acid sequence and BpCol2-like nucleic acid sequences, which are related to CONSTANS (CO) gene family, and gene products thereof as well as methods and use therein for influencing growth of a plant or roots of a plant. Particularly the invention is related to cloning and expression of BpCol2 gene in plants. The invention is further related to DNA constructs, vectors, transgenic cells, plants and seeds comprising said nucleic acid sequences and gene products.

### TECHNICAL FIELD OF THE INVENTION

The present invention is related to the genetic control of influencing of growth in plants and the cloning and expression of genes therein. Particularly the invention is related to cloning and expression of BpCol2 gene of birch (Betula pendula) and nucleic acid sequences and their gene products as well as BpCol2-like nucleic acid molecules from birch and other species. The invention is further related to the transgenic cells, plants and seeds and use thereof in forestry, agriculture and horticulture.

### BACKGROUND OF THE INVENTION

An understanding of the genetic mechanisms of regulation of growth in plants provides a means for altering the growth characteristics of the target plant. Especially the regulation of root growth is important in crop plants, horticultural plants and trees whose root length may be controlled.

Transgenic trees have been produced with altered properties. WO 00/12715 relates to the modification of the morphology of plant fiber cells using an expansin gene. Increased fiber length leads to the production of paper with increased strength. Short fibers are suitable in some cases such as in the production of smooth-surfaced papers. In practice short and long fibers from different species are mixed to get optimal material for processor. In woody plants fibers are made from dead cell wall material. Long fibers include longer living cells during growth, before fiber formation.

WO 01/66777 is related to the transgenic trees with improved properties such as increased growth, biomass production and xylem fiber length using the modification of the level of a plant hormone, gibberellin, in trees.

WO 00/32780 discloses a FLOWERING LOCUS F (FLF) gene and nucleic acid molecules for altering the flowering time of a plant. U.S. Pat. No. 6,077,994 and WO 96/14414 disclose the CONSTANS (CO) gene, also called FG, of Arabidopsis thaliana for influencing flower characteristics in transgenic plants, especially the timing of flowering. Arabidopsis CONSTANS (CO) gene promotes flowering under long day (LD) photoperiod conditions but does not affect flowering under short days (SD) (Putterhill, et al., Cell 80:847-857, 1995). The production of seed of any kind is very dependent upon the ability of the plant to flower, to be pollinated and to set seed. In horticulture, control of the timing of flowering is important.

Related technology is disclosed in WO 00/71722, EP 1033405 and in the publication of Yuceer et al. (Plant Science 163:615-625, 2002).

It has been shown that plants carrying mutations of CO gene flower later than their wild-types under long days but at the same time under short days the CO gene product is involved in the promotion of flowering. A total of 16 CO-homologs in the Arabidopsis genome have been identified but their function remains to be elucidated (Robson, et al., Plant Journal 28:619-631, 2001). The two CONSTANS like genes so far tested, COL1 and COL2, in transgenic plants had little effect on flowering time (Ledger et al., Plant Journal 26:15-22, 2001). The CO protein contains an arrangement of cysteins at the amino acid end of the protein that is characteristics of zinc fingers, that probably binds DNA and acts as a transcription factor.

A clear need exists for a method for influencing the plant growth especially the decreasing or promoting the growth of the roots of a plant.

### SUMMARY OF THE INVENTION

The present invention is related to the genetic control of regulation of growth in plants and the cloning and expression of genes therein. Particularly the invention is related to cloning and expression of BpCol2 gene of birch (Betula pendula) and nucleic acid sequences and their gene products as well as BpCol2-like nucleic acid molecules from birch and other species. The invention is filter related to the transgenic cells, plants and seeds and use therein.

The present invention is related to isolated nucleic acid molecules for influencing growth of plants, especially growth of plant roots, characterized in that the nucleic acid molecules comprise nucleotide sequences capable of encoding polypeptides which comprise an amino acid sequence of SEQ ID NO:2 or SEQ ID NO:3, SEQ ID NO:4, SEQ ID NO:5, SEQ ID NO:6 or SEQ ID NO:7 and derivatives and fragments thereof and said nucleic acid sequences have the capacity of regulating the length of a plant or roots of a plant.

Preferably nucleic acids according to the present invention comprise nucleotide sequences capable of encoding polypeptides which comprise an amino acid sequence of SEQ ID NO:2, SEQ ID NO:3, SEQ ID NO:4, SEQ ID NO:5, SEQ ID NO:6 or SEQ ID NO:7 of Betula pendula and said nucleic acid sequences have the capacity of influencing the growth of a plant or roots of a plant.

Nucleic acid molecules according to the present invention are BpCol2-like nucleic acid sequences capable of encoding BpCol2-like gene products, which are substantially homologous to the gene product of CONSTANS cDNA and said BpCol2-like nucleic acid sequences have the capacity of influencing the growth of a plant or roots of a plant.

Preferably the amino acid sequence of SEQ ID NO:2 is encoded by a nucleic acid sequence of SEQ ID NO: 1 of Betula pendula.

Thus an objective of the present invention is to provide isolated nucleic acid molecules, new methods and means for influencing the growth of plants, preferably the growth of plant roots.

Another embodiment of the present invention is to provide means for carrying out said methods. The means are BpCol2 cDNA and BpCol2-like nucleic acid sequences, fragments and derivatives thereof as well as their complementary strands and BpCol2 gene products expressed by the BpCol2 nucleic acid sequences of the present invention.

An embodiment of the present invention is a method of influencing growth in a plant, characterized in that said method comprises the steps of introducing at least one nucleic acid molecule according to any of claims 1 to 8 into a plant cell, optionally such that expression of the nucleic acid molecule is under the control of a promoter, and overexpressing the nucleic acid molecule and regenerating a plant from said transformed plant cell, wherein the introduced DNA is expressed in the transformed plant. Preferably the growth of the roots is decreased by using an overexpressing sense gene.

An embodiment of the present invention comprises a DNA construct for cloning and/or transforming plants, said DNA construct comprising one or more nucleic acid sequences of the invention functionally combined with regulatory sequences. A vector, a host cell, a transformed plant and seeds comprise the nucleic acids of the present invention.

An embodiment of the present invention is a method of influencing growth in a plant, characterized in that said method comprises the step of introducing at least one nucleic acid molecule according to any of claims 1 to 8 in an anti-sense orientation into a plant cell, optionally such that expression of the nucleic acid molecule is under the control of a promoter and regenerating a plant from said transformed plant cell, wherein the introduced DNA is expressed in the transformed plant. Preferably the growth of the roots is promoted by using an antisense gene.

An embodiment of the present invention is a method of influencing growth in a plant, characterized in that the method comprises the steps of stably incorporating into a plant genome a DNA construct comprising a promoter and a nucleic acid coding sequence encoding gene capable of modifying the length of the plant or length of roots and regenerating a plant having an altered genome.

Another embodiment of the present invention is to use BpCol2 gene products, fragments and derivatives thereof for technically modifying the expression of a gene or a modified gene/a natural variant of the gene to alter the growth of a plant.

Another embodiment of the present invention is to use the nucleic acid molecules of the present invention and polypeptides encoded by them in forestry for regulating the length of roots of a plant.

Another embodiment of the present invention is to use of the nucleic acid molecules of the present invention and polypeptides encoded by them in agriculture and horticulture for regulating the length of roots of a plant.

Another embodiment of the present invention is to use of the nucleic acid molecules according to any of claims for photoperiodic control of the vegetative growth in plants.

The present invention provides isolated nucleic acid sequences comprising SEQ ID NO: 1 and nucleic acid sequences encoding polypeptides of SEQ ID NO:2, SEQ ID NO:3, SEQ ID NO:4, SEQ ID NO:5, SEQ ID NO:6 or SEQ ID NO:7.

The characteristic features of the present invention are defined in detail in the claims.

### A SHORT DESCRIPTION OF THE DRAWINGS

FIG. 1A depicts the ligation of BpCol2 transgene in sense orientation to pDE1001 vector (Stratagene). The transgene was placed under the 35S promoter. Recognition sites for restriction enzymes HindIII and SacI are shown.

FIG. 1B depicts the ligation of BpCol2 transgene in antisense orientation to pDE1001 vector (Stratagene). The transgene was placed under the 35S promoter. Recognition sites for restriction enzymes HindIII and SacI are shown.

FIG. 2 depicts the amino acid sequence comparison between birch BpCol2 (marked as BpCOL2 in the alignment) and Arabidopsis CONSTANS (marked as AtCO in the alignment) sequences carried out using GeneStream align (Pearson et al., Genomics 46: 24-36, 1997). 366 amino acids vs. 355 amino acids were aligned, gap penalties: −12/−2, the sequence identity was 43.5%. Global alignment score: 899. Pfam-program analysis identified two B-box regions and a CCT-domain in the BpCol2 protein. CCT domain is located in the carboxyl terminal part of a polypeptide and two zinc-finger domains (B-box-domains) are located near the amino terminus. The B-Box and CCT-domains have been marked to the sequences with underlines. The area between B-Boxes and CCT domain is different between BpCol2 and CONSTANS. This might suggest functional differences between BpCol2 and CONSTANS genes.

FIG. 3 depicts the multiple sequence alignment of amino acid sequences homologous to Arabidopsis CONSTANS—and identified from birch ESTs: BpCol2 (SEQ ID NO:2), BpCol1 (SEQ ID NO:3), BpCol3 (SEQ ID NO:4), BpCol4 (SEQ ID NO:5), BpCol5 (SEQ ID NO:6) and BpCol11 (SEQ ID NO:7). The alignment of BpCol1-4 was done using Clustal W (1.82). All sequences of BpCol proteins have been analyzed with Pfam-program. These sequences are CONSTANS related sequences including a CCT domain in the carboxyl terminal part of a polypeptide and one or two zinc-finger domains (B-box-domains) near the amino terminus. The B-Box and CCT-domains of BpCol proteins have been marked to the sequences with underlines except the B-box and CCT domains of BpCol5 and 11 which have only been analyzed with Pfam-program. The two B-box domains of BpCol2, BpCol3 and BpCol11 are located between amino acids 19-107, 3-91 and 39-126, respectively. One B-Box domain of BpCol4 and BpCol5 is located between amino acids 16-61 and 16-61. The amino terminal sequence of BpCol1 is not cloned yet. Thus the B-Box domains of BpCol1 have not been identified. However, BpCol1 is closely related to the CONSTANS of A. thaliana and the BpCOL1 has the CCT-domain which has been shown to be an important domain for the function of the CONSTANS protein family. The CCT domain of BpCol1, BpCol2, BpCol3, BpCol4, BpCol5 and BpCol11 genes are located between amino acid 157-195, 299-337, 274-312, 372-410, 350-388 and 363-401, respectively.

FIG. 4A depicts the flowering time as the amount of rosette leaves of T2 generation BpCol2 transgenic Arabidopsis lines grown in a growth room in short day (SD) conditions. Flowering time of T₂ plants was measured when the first visible flower bud was visible. 7-9 plants/line were tested. The line tested is shown along the horizontal axis and the amount of rosette leaves are shown on the vertical axis. Data is presented as mean±t.SE (P=0.05). The lines assayed were high-expressing sense lines (s25 and s40), antisense lines (as17 and as18) and vector control line (vc2). The amount of rosette leaves of BpCol2 overexpressing lines was higher than the amount of rosette leaves of vector control. In contrast, the amount of rosette leaves of antisense lines was smaller than the amount of rosette leaves of vector control. Abbreviations: vc, vector control; s, sense; as, antisense.

FIG. 4B depicts the flowering time of T₂ generation transgenic Arabidopsis plant lines grown in short day (SD) conditions. Flowering time of T₂ plants was measured when the first visible flower bud was visible. 7-9 plants/line were tested. The line tested is shown along the horizontal axis and the days to flowering are shown on the vertical axis. Data is presented as mean±t.SE (P=0.05). The lines assayed were high-expressing sense lines (s25 and s40), antisense lines (as17 and as18) and vector control line (vc2). The antisense line as17 flowered at 35 days±t.SE 2.2 and as18 line at 34 days±t.SE 2.5 which is only slightly earlier than vector control line vc2 which flowered at 42 days±t.SE 1.9. The effect on flowering was clearer with overexpressing lines s25 and s40 which flowered at 50 days±t.SE 2.7 and 52 days±t.SE 3.2, respectively. Abbreviations: vc, vector control; s, sense construct; as=antisense.

FIG. 5A depicts the flowering time as the amount of rosette leaves of T2 generation BpCol2 transgenic Arabidopsis lines in a growth room in long day (LD) conditions. Flowering time was measured when first visible flower bud was visible. 7-9 plants/line were tested. The line tested is shown along the horizontal axis and the days to flowering are shown on the vertical axis. Data is presented as mean±t.SE (P=0.05). The lines assayed were high-expressing sense lines (s25 and s40), antisense lines (as17 and as18) and vector control line vc2. There was no effect with antisense lines. In contrast, the amount of rosette leaves of BpCol2 overexpressing lines was higher than the amount of rosette leaves of vector control. Abbreviations: vc, vector control; s, sense; as, antisense.

FIG. 5B depicts the flowering time as days of T2 generation BpCol2 transgenic Arabidopsis lines in a growth room in long day (LD) conditions. Flowering time was measured when first visible flower bud was visible. 7-9 plants/line were tested. The line tested is shown along the horizontal axis and the days to flowering are shown on the vertical axis. The lines assayed were high-expressing sense lines (s25 and s40), antisense lines (as17 and as18) and vector control line vc2. There was no effect with antisense lines. In contrast, overexpressing lines s25 and s40 flowered at 29 days±t.SE 1.3 and 33 days±3.4 t.SE, respectively. Vector control flowered at 24 days±t.SE 0.98. Data is presented as mean±t.SE (P=0.05). Abbreviations: ve, vector control; s, sense.

FIG. 6A depicts the flowering time as the amount of rosette leaves of T3 generation BpCol2 transgenic Arabidopsis lines in a greenhouse in short day (SD) conditions. Abbreviations: vc, vector control; s, sense.

FIG. 6B depicts flowering time as days of T3 generation BpCol2 transgenic Arabidopsis lines in a greenhouse in long day (SD) conditions. Abbreviations: vc, vector control; s, sense.

FIG. 7A depicts the flowering time as the amount of rosette leaves of T3 generation BpCol2 transgenic Arabidopsis lines in a greenhouse in long day (LD) conditions. Abbreviations: vc, vector control; s, sense.

FIG. 7B depicts flowering time as days of T3 generation BpCol2 transgenic Arabidopsis lines in a greenhouse in long day (LD) conditions. Abbreviations: vc, vector control; s, sense.

FIG. 8 depicts phenotypes of 51-day old transgenic Arabidopsis plant lines of T2 generation grown in short day (SD) conditions (12 h light/12 h dark) and having sense BpCol2 (left), vector control (middle) or antisense BpCol2 (right) transgene. The plants having overexpressing BpCol2 gene in sense orientation flowered later than the plants having a vector control in short day (SD) conditions. In contrast, a plant line containing an antisense line flowered earlier than the vector control line. Even if the development of overexpressing lines was slower, the color and the shape of the leaves were normal and there were no clear visible changes in plant morphology in those parts of a plant which were above soil. No clear visible changes other than the flowering time with antisense lines were seen.

FIG. 9 depicts phenotypes of 30 days old transgenic Arabidopsis plant lines of T2 generation in long day (LD) conditions (16 h light, 8 h dark). The plant in left carries BpCol2 transgene in sense orientation. The plant in right carries a vector control. Overexpressing BpCol2 gene flowered later than vector control. The shape and size of the leaves of the overexpressing line were normal. The effect of 35S::BpCol2 transgene on flowering time was not so clear in LD than in SD. However, there is an late flowering phenotype both in SD and in LD. This suggests that the BpCol2 overexpressing lines maybe insensitive to the daylenght.

FIG. 10 depicts the flowering phenotype of T3 generation BpCol2 Arabidopsis plants grown in long day (LD) conditions. 1) vector control; 2) antisense; 3) sense.

FIG. 11 depicts the phenotypes of transgenic Arabidopsis plants: 1) antisense BpCol2 plants (left), 2) vector control (middle) and 3) sense BpCol2 plants (right). Root lengths were photographed from 7 days old T₂ generation plants grown in short day (SD) conditions. The plant carrying an antisense construct had longer roots and the plant carrying an overexpressing (sense) line had shorter roots than the vector control. This is somewhat surprising considering the heterologous nature of the antisense-construct used. However, it strenghtens our view that this phenomena is not restricted to birch e.g. it is not birch-specific, but is applicable to plants in general. Furthermore, it points towards the fact that there is a functionally and structurally conserved CONSTANS-like gene(s) in Arapidopsis, the down regulation of which by using more specific constructs with its own sequences, alone or in combination, leads to the same and even stronger phenotypic changes as seen with the BpCOL2 gene. Birch BpCol2 represses root growth in Arabidopsis.

FIG. 12 depicts the root phenotype of transgenic T3 generation of BpCol2 overproducing Arabidopsis lines grown in short day (SD) conditions, from left vector control and sense.

FIG. 13 depicts the root phenotype of transgenic T3 generation BpCol2 overproducing Arabidopsis plants grown in long day (LD) conditions, from left vector control and sense.

FIG. 14 depicts the root lengths (mm) of BpCol2 overproducing transgenic Arabidopsis plants grown in short day (SD) conditions. Abbreviations: vc, vector control; s, sense.

FIG. 15 depicts the root lengths (mm) of BpCol2 overproducing transgenic Arabidopsis lines grown in long day (LD) conditions. Abbreviations: vc, vector control; s, sense.

FIG. 16 depicts the root growth of BpCol2 transgenic birches in long day (LD) conditions. The root lengths were measured with transparent ruler from 4 weeks old plants grown in LD. The roots of the overexpression lines were shorter than in wild type plants. In contrast, antisense construct had a positive effect on root growth. The lengths of the roots of the overexpression lines 11 and 15 were 2.1 cm±t.SE 0.7 and 2.3 cm±t.SE 0.36, respectively. The root length of the antisense line 5 was 3.7 cm±t.SE 0.43 whereas the root length of the wild type plant was 3.4 cm±t.SE 0.66. It is a well-known fact for those skilled in the art that using the method of expressing a gene in antisense orientation for the purpose of down regulation of gene expression may lead to greater variation in response between the different lines due to the relative inefficiency of the method. Thus, the use of alternative gene silencing methods, like RNAi (double stranded-RNA expression constructs) or siRNA (small interfering-RNA) is expected to lead to a more pronounced effects more reliably. Data was presented as mean±t.SE (P=0.05).

FIG. 17 depicts the phenotypes of transgenic birches: 1) antisense BpCol2 (left), 2), 3) sense BpCol2 (two in the middle) and 4) wild type (right). Phenotypes of BpCol2 transgenic birch roots in LD. Plants were rooted 4 weeks in LD in in vitro growth room. The plants containing an antisense construct had longer roots and overexpression lines had shorter roots compared to wild type plant.

FIG. 18 depicts the phenotypes of two month-old transgenic birches carrying a BpCol2 transgene grown in long day (LD) conditions on soil. The plants are shown from left to right: 1) wild type birch, 2) antisense 5, 3) sense 20, 4) sense 15, 5) sense 11. The plant line carrying an antisense BpCol2 grew faster than the wild type plant whereas overexpressing lines grew more slowly than the wild type plant in long day (LD) conditions. The plant line 11 carrying a sense gene had the slowliest growing phenotype but the plant lines 15 and 20 with sense genes had also slowly growing phenotype.BpCol2 controls growth in birch.

FIG. 19A depicts the root tip morphology of a BpCol2 transgenic Arabidopsis plant transformed with a sense construct and grown in long day (LD) conditions (16 h). 7 days old plants were transferred onto the microscope slides and photographed with light microscope (20× magnification). The meristematic (MZ), elongation (EZ) and differentiation zones (DZ) are indicated. In the root tip the of BpCol2 overexpressing line the cell morphology was altered compared to vector control including smaller cell size. In addition, the BpCol2 overexpressing line may have shortened elongation zone and alterations in meristematic zone including the lack of root cap compared to vector control.

FIG. 19B depicts the root tip morphology of a BpCol2 transgenic Arabidopsis plant transformed with a vector control and grown in long day (LD) conditions (16 h) 7 days old plants were transferred onto the microscope slides and photographed with light microscope (20×magnification). The meristematic (MZ), elongation (EZ) and differentiation zones (DZ) are indicated.

FIG. 20A depicts the root tip morphology of a BpCol2 transgenic Arabidopsis plant transformed with a sense construct and grown in short day (SD) conditions (10 h). The elongation region of the root tip is shorter than in the control plant. 7 days old plants were transferred onto the microscope slides and photographed with light microscope (20×magnification). The meristematic (MZ), elongation (EZ) and differentiation zones (DZ) are in indicated. The changes in the cell structures of BpCol2 overexpressing line were more pronounced in short day than in long day. The thin structure of the BpCol2 overexpressing line was more clear and the cells are shorter in SD compared to LD. Especially BpCol2 overexpressing line may have more clearly shortened elongation zone in SD than in LD. There might be alterations of BpCol2 overexpression line in meristematic zone including the lack of root cap compared to vector control in SD

FIG. 20B depicts the root tip morphology of a BpCol2 transgenic Arabidopsis plant transformed with a vector control and grown in short day (SD) conditions (10 h). 7 days old plants were transferred onto the microscope slides and photographed with light microscope (20×magnification). The meristematic (MZ), elongation (EZ) and differentiation zones (DZ) are in indicated.

FIG. 21A depicts the root cell lengths from differentiation zone of a BpCol2 transgenic Arabidopsis plant transformed with a sense construct and grown in long day (LD) conditions (16 h). 7 days old plants were transferred onto the microscope slides and photographed with light microscope (20×magnification). Cell lengths of BpCol2 transgenic lines were shorter compared to vector control in LD.

FIG. 21B depicts the root cell lengths from differentiation zone of a BpCol2 transgenic Arabidopsis plant transformed with a vector control and grown in long day (LD) conditions (16 h). 7 days old plants were transferred onto the microscope slides and photographed with light microscope (20×magnification).

FIG. 22A depicts the root cell lengths from differentiation zone of a BpCol2 transgenic Arabidopsis plant transformed with a sense construct and grown in long day (SD) conditions (10 h). 7 days old plants were transferred onto the microscope slides and photographed with light microscope (20×magnification). Cell lengths of BpCol2 transgenic lines were shorter compared to vector control in SD.

FIG. 22B depicts the root cell lengths from differentiation zone of a BpCol2 transgenic Arabidopsis plant transformed with a vector control and grown in long day (SD) conditions (10 h). 7 days old plants were transferred onto the microscope slides and photographed with light microscope (20×magnification).

FIG. 23 depicts the northern expression analysis of overexpressing Arabidopsis lines s25 and s40 in short day (SD) conditions. The expression levels of T₂ (vc2, s25 and s40 on the left) and T₃ (vc2, s25 and s40 on the right) generation plants are shown. The expression levels of T2 and T3 generation plants are at the same level. Abbreviations: s, sense; vc, vector control.

FIG. 24A depicts the root branching phenotype of a 14 days old BpCol2 transgenic overexpressing Arabidopsis plant grown in short day (SD) conditions (12 h). 35S:BpCol2 transgene in sense orientated plants may induce the changes in apical dominance. This could explain the strong formation of secondary roots and the weak growth of primary root.

FIG. 24B depicts the root branching phenotype of a 14 days old Arabidopsis control plant grown in short day (SD) conditions (12 h). In vector control the strong primary root and the smaller secondary roots were clearly seen.

FIG. 25 depicts growth cessation of BpCol2 transgenic birch lines in SD conditions (12 h).

BpCol2 overexpressing lines are s11, s15 and s20 and antisense line is as5 (s=sense, as =antisense, wt=wild type). For the growth cessation experiment plants were transferred from LD to the SD (12 h light/12 h dark) and the cessation of the growth in SD was followed regularly during days 0, 3, 5, 7, 8, 10, 12, 14 and 16. BpCol2 overexpressing lines s11 and s15 did not response to short day and were insensitive to 12 h photoperiod. Data was presented as mean±t.SE (P=0.05). 4 plants/line were studied.

FIG. 26 depicts the total growth and days to growth cessation of transgenic BpCol2 birches in short day (SD) conditions.

### THE DETAILED DESCRIPTION OF THE INVENTION

Definitions

The terms used in the present invention have the meaning they have in the fields of molecular biology, botany, genetics, including recombinant DNA technology. Some terms in the present invention are, however, used with a somewhat deviating or broader manner. Accordingly, in order to avoid uncertainty caused by terms with unclear meaning some of the terms used in this specification and in the claims are defined in more detail below.

The term “BpCol2” means a birch (Betula pendula Roth) BpCol2 gene related to constans family and affects growth in Arabidopsis and birch and flowering in Arabidopsis. BpCol2 is also called as KKO2. The CO protein contains an arrangement of cysteins at the amino acid end of the protein that is characteristics of zinc fingers, that probably binds DNA and acts as a transcription factor.

The term “BpCol2-like compounds” means “BpCol2” or “BpCol2-like proteins” but also includes nucleic acid sequences encoding said BpCol2 or BpCol2-like proteins

The term “BpCol2-like compounds” means compounds, which act as BpCol2-like proteins. They include polypeptides “substantially homologous” at amino acid level having a significant similarity or identity of at least about 60%, more preferred embodiments include at least 65%, 70%, 75%, 80%, most preferably more than 85% identity with the reference sequence of SEQ ID NO:2, SEQ ID NO:3, SEQ ID NO:4, SEQ ID NO:5, SEQ ID NO:6 and SEQ ID NO:7.

The term “BpCol2-like compounds” means protein molecules or polypeptides being substantially homologous to BpCol2 at amino acid level. Said “BpCol2-like molecules” are obtainable by isolation from natural sources. The BpCol2-like molecules are also producible by synthetic, semisynthetic, enzymatic and other biochemical or chemical methods including recombinant DNA techniques.

The term “BpCol2-like compounds” also comprises polypeptides having the structure, properties and functions characteristic of BpCol2-like proteins, including BpCol2-like proteins, wherein one or more amino acid residues are substituted by another amino acid residue. Also truncated, complexed or chemically substituted, forms of said BpCol2-like proteins are included in the term. Chemically substituted forms include for example, alkylated, esterified, etherified or amidized forms with a low substitution degree, especially using small molecules, such as methyl or ethyl, as substituents, as long as the substitution does not disturb the properties and functions of the BpCol2-like proteins. The truncated, complexed and/or substituted variants of said polypeptides are producible by synthetic or semisynthetic, including enzymatic and recombinant DNA techniques. The only other prerequisite is that the derivatives still are substantially homologous with and have the properties and/or express the functions characteristic of BpCol2-like proteins.

The term “BpCol2-like compounds” otherwise covers all possible splice variants of BpCol2. The BpCol2-like compounds can exist in different isoforms or allelic forms.

More specifically “BpCol2-like proteins” are substantially homologous with the amino acid sequence SEQ ID NO:2, SEQ ID NO:3, SEQ ID NO:4, SEQ ID NO:5, SEQ ID NO:6 or SEQ ID NO:7.

The term “isoform” refers to the one of several forms of the same protein, whose amino acid sequences differ slightly but whose general activity is identical. “Isoforms” may originate from different sources, e.g. different plant species. Isoforms of BpCol2 compounds can be generated by the cleavage. Different enzymatic and non-enzymatic reactions, including proteolytic and non-proteolytic reactions, are capable of creating truncated, derivatized, complexed forms of BpCol2 proteins.

In the present invention the term “BpCol2-like compounds” includes nucleic acid sequences, which belong to the active BpCol2-like compounds of the present invention and which comprise isolated or purified “nucleic acid sequences” encoding BpCol2-like proteins or nucleic acid sequences with substantial similarity. They can be used as such or introduced into suitable transformation or expression vectors, which in turn can be introduced into suitable host organism to provide prokaryotic, eukaryotic organisms as well as transgenic plants capable of expressing altered levels of BpCol2-like proteins.

The term “nucleic acid sequences” refers to single or double-stranded polymer of deoxyribonucleotide or ribonucleotide bases. It includes chromosomal DNA, self-replicating plasmids, polymers of DNA or RNA. The “nucleic acid sequences” of the present invention are not in their natural state but are isolated and purified from their natural environment as transiently expressed mRNAs from a tissue. Thereafter the mRNAs are purified and multiplied in vitro in order to provide by technical means new copies, which are capable of encoding said BpCol2-like proteins. The nucleic acid sequences include both genomic sequences and cDNA.

The term “genomic sequence” means the corresponding sequence present in the nucleus of the plant cells and comprising introns as well as exons. In the present context the term “cDNA” means a DNA sequence obtainable by reversed translation of mRNA translated from the genomic DNA sequence including the complementary sequence.

The term “nucleic acid sequence encoding BpCol2 or BpCol2-like proteins” means nucleic acid sequences encoding BpCol2 or substantially homologous sequences. Said sequences or their complementary sequences or nucleic acid sequences containing said sequences or parts thereof, e.g. fragments truncated at the 3′-terminal or 5′-terminal end, as well as such sequences containing point mutations, are especially useful for as probes, primers and for preparing DNA constructs, plasmids and/or vectors useful for modulating the level of expression in plant tissues.

It is however clear for those skilled in the art that other nucleic acid sequences are capable of encoding BpCol2-like proteins and useful for their production can be prepared. Said nucleic acid sequences and/or their complementary sequences should be capable of hybridizing under highly stringent condition (Sambrook and Russell, 2001) with SEQ ID NO:1, nucleic acid sequences encoding polypeptides shown in SEQ ID NO:2, SEQ ID NO:3, SEQ ID NO:4, SEQ ID NO:5, SEQ ID NO:6 or SEQ ID NO:7.

The nucleic acid sequences of the present invention should have a substantial similarity with the sequences encoding BpCol2 or BpCol2-like proteins. “Substantial similarity” in this context means that the nucleotide sequences fulfill the prerequisites defined above and have a significant similarity, i.e. a sequence identity of at least 40%, more preferred embodiments include at least 50%, 55%, 60%, 65%, 70%, 75%, 80%, most preferably more than 85% with the reference sequence.

The term “nucleic acid sequences encoding BpCol2 or BpCol2-like proteins” include their truncated or complexed forms as well as point mutations of said nucleic acid sequences as long as they are capable of encoding amino acid sequences having the essential structural features as well as the properties and/or functions of said BpCol2-like compounds.

The nucleic acid sequences are useful as such or inserted in transformation or expression vectors or host, said nucleic acid sequences being capable of encoding BpCol2 or BpCol2-like proteins which are recognizable by binding substances specifically recognizing said BpCol2 or BpCol2-like proteins.

The BpCol2-like compounds include in addition to the proteins and nucleic acid sequences also binding substances.

The term “binding substances” means substances, which are capable of recognizing and specifically binding to natural BpCol2 and/or BpCol2-like proteins or at least one specific portion of said molecules. Such binding substances are for example antibodies, receptors or ligands or proteins, specifically recognizing or binding to BpCol2 or BpCol2-like proteins, ligands of BpCol2-like proteins or other binding proteins or peptides, comprising e.g. specific portions of said BpCol2-like compounds, but above all they mean antibodies capable of specifically recognizing one or more BpCol2-like compounds alone or in any combination. The antibodies include both polyclonal and/or monoclonal antibodies as well as fragments or derivatives thereof. Preferably, such binding substances recognize and bind to specific epitopes or active sites of the BpCol2-like compounds.

Said “binding substances” can be produced using specific domains of BpCol2-like compounds, their isomers as well as their fragments, derivatives and complexes with the prerequisite that they are capable of functioning in respective signaling pathway.

The General Description of the Invention

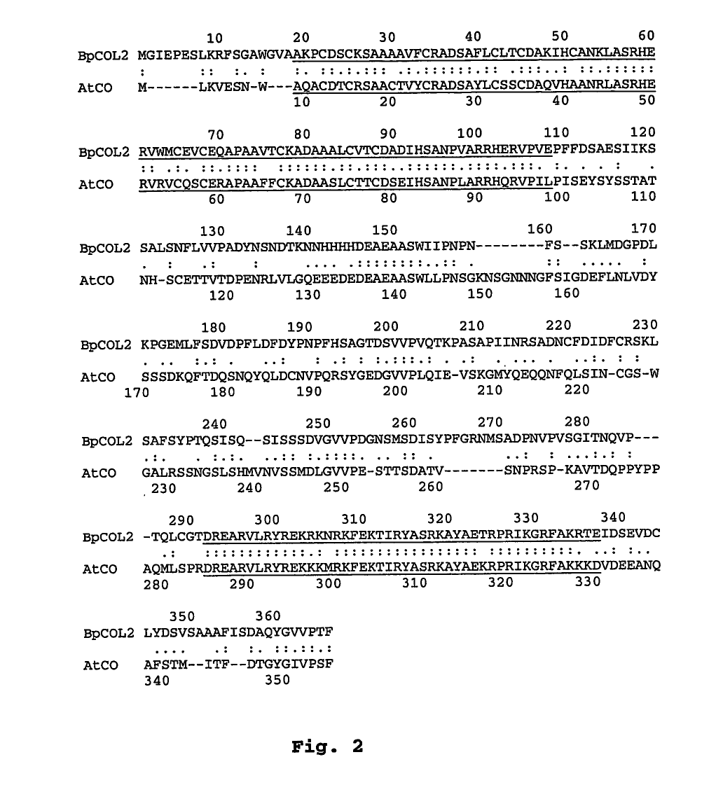

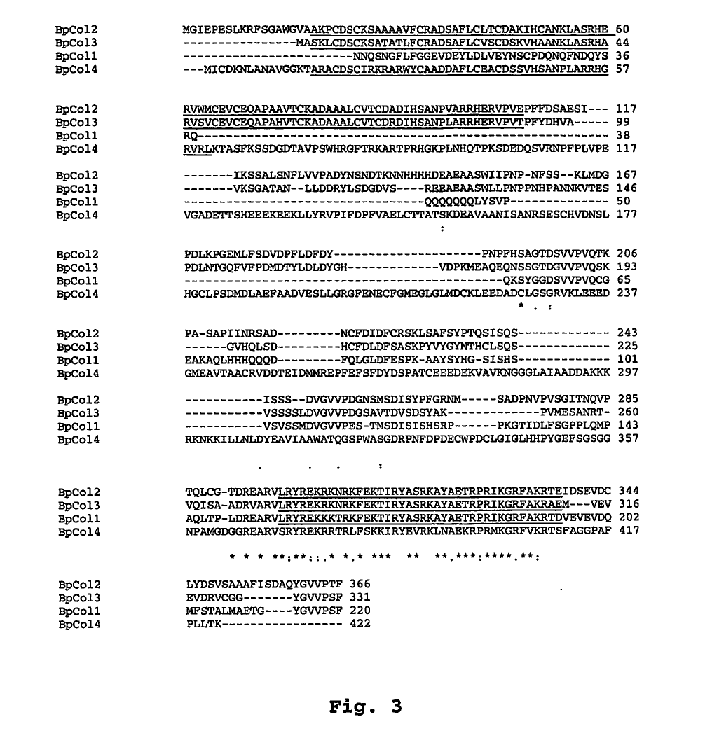

The inventors of the present invention unexpectedly found that BpCol2 gene (SEQ ID NO:1) isolated from birch (Betula pendula L. Roth) has an effect on the growth of a plant, especially Arabidopsis and birch. The birch BpCol2 gene and its homologs (FIG. 3) are related to CONSTANS family (FIG. 2) and affects growth in Arabidopsis and birch and flowering in Arabidopsis. It was unexpectedly noticed that BpCol2 is a negative regulator of growth in plants.

The present inventors have used birch (Betula pendula Roth) as a model for studying photoperiodic regulation of growth cessation in trees. An EST (Expressed Sequence Tag) library of birch was constructed and several genes related to the CONSTANS family were found.

Sequence comparison to known sequences in databases revealed that that isolated sequences are related Arabidopsis CONSTANS gene and include a CCT domain in the terminal part of a polypeptide and two zinc-finger domains near the amino terminus.

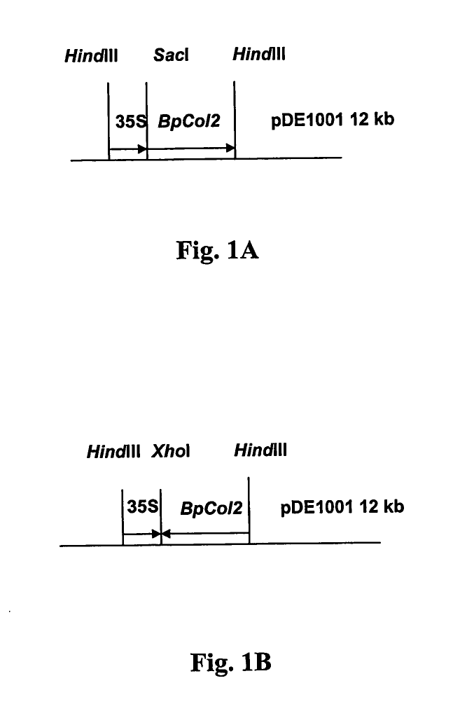

Transgenic Arabidopsis and birch plants carrying BpCol2 gene in sense or antisense orientation under CaMV 35S promoter (FIGS. 1A and 1B) in the binary vector pDE1001 (Denecke, et al. 1992, EMBO J. 11:2345-2355) were made using Agrobacterium tumefaciens-mediated gene transfer. Whole plants (T₀ generation) of Arabidopsis ecotype Col were transformed by dipping method (Clough, et al. Plant Journal. 16:735-743, 1998) (Example 2). Kanamycin resistant plants were selected in the T₂ generation. T₃ plants homozygous for the BpCol2 transgene were also used for the flowering time, expression analysis and root growth experiments. Transgenic Arabidopsis thaliana lines S25 and S40 contained the sense 35S::BPCOL2 construct and the lines AS17 and AS18 contained the antisense 35S::BPCOL2 transgene. Lines VC2, VC7 and VC12 contained the vector control construct 35S::leader sequence.

The leaves and shoots of birch ecotype JR 1/4 were transformed using a birch transformation protocol (Keinonen-Mettala, et al. Plant Cell Reports. 17:356-361, 1998) (Examples 5 and 7). Homozygotes with a single active nptII locus were selected based on the segregation of kanamycin resistance. Transgenic birch lines S11, S15 and S20 contained the sense 35S::BPCOL2 construct and the line AS5 contained the antisense 35S::BPCOL2 transgene. A wild type birch was used as a control.

Transgenic Arabidopsis plants overexpressing a birch gene BpCol2 flowered later than control plants including only vector control whereas transgenic plants carrying BpCol2 gene in antisense orientation flowered earlier. This suggests that this gene may act as a repressor of flowering in Arabidopsis.

Flowering time of Arabidopsis plants was measured under defined conditions by growing plants in short day (SD) or long day (LD) conditions in in vitro culture rooms. A short day comprised a photoperiod of 12 hours or 10 hours and a long day (LD) photoperiod was 16 hours light. Flowering time was measured by counting the number of rosette leaves at the time point when the first flower was visible (Example 3).

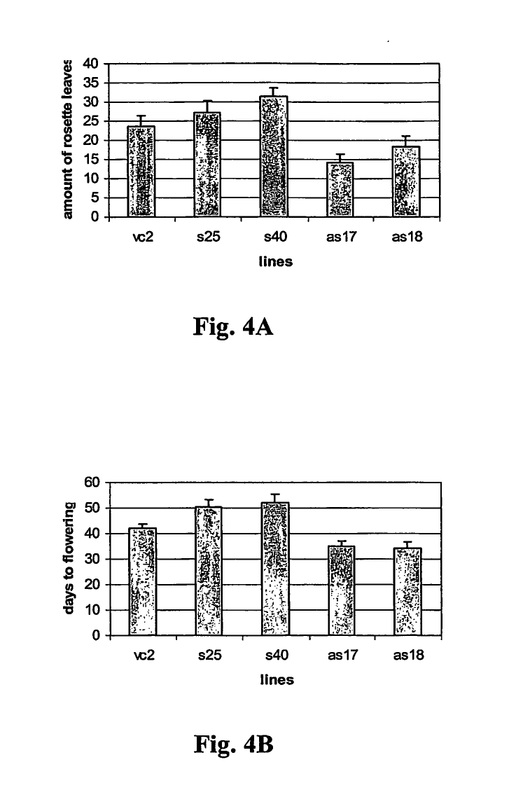

Transgenic Arabidopsis plants carrying and overexpressing a BpCol2 gene in sense orientation flowered later than the plants carrying a vector control in short day (SD) conditions. In contrast, a plant line containing an antisense BpCol2 gene flowered earlier than the plant carrying a vector control line (FIGS. 4A and 4B). Even if the development of overexpressing lines was slower, the color and the shape of the leaves were normal and there were no clear visible changes in plant morphology in those parts of a plant which were above soil. No clear visible changes other than flowering time with antisense lines were seen.

Flowering time of T₂ generation transgenic Arabidopsis plant lines grown in short day (SD) conditions was measured when first flower bud was visible (FIGS. 4A and 4B). The effect on flowering was clearest with overexpressing lines s25 and s40 which flowered at 50 days and 52 days.

Transgenic Arabidopsis plants overexpressing BpCol2 gene flowered later than vector control in long day (LD) conditions. The shape and size of the leaves of the overexpressing line were normal. The effect of 35S:BpCol2 transgene on flowering time was not so clear in long day conditions (LD) than in short day (SD) conditions. However, there is a late flowering phenotype both in SD and in LD. This suggests that the BpCol2 overexpressing lines maybe insensitive to the day length.

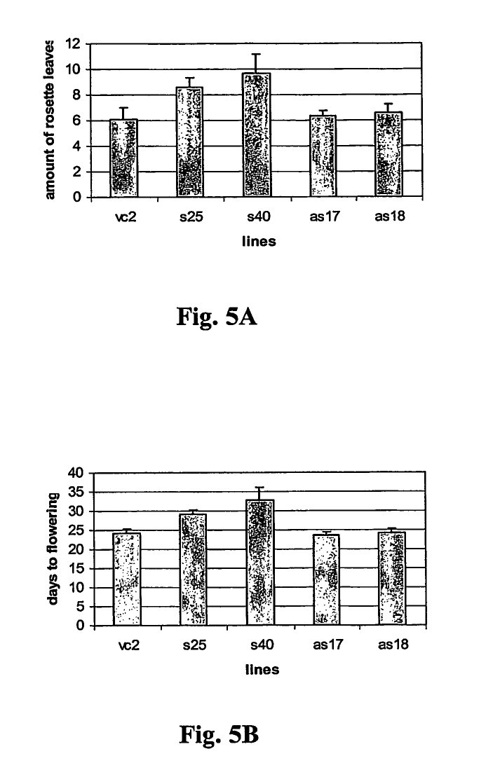

Flowering time of T₂ generation transgenic Arabidopsis plants in long day (LD) conditions was measured when first flower bud was visible (FIG. 5B). There was no effect with antisense lines. In contrast, overexpressing lines s25 and s40 flowered at 29 days±t.SE 1.3 and 33 days±3.4 t.SE, respectively. Vector control flowered at 24 days±t.SE 0.98.

The flowering time of the T₃ generation transgenic BpCol2 Arabidopsis plants grown in greenhouse in long day (LD) and short day (SD) conditions was measured as the amount of rosette leaves or as days from sowing. The flowering time of the T₂ generation transgenic BpCol2 Arabidopsis plants grown in controlled growth room in long day (LD) and short day (SD) conditions was measured as the amount of rosette leaves or as days from sowing.

The amount of rosette leaves correlates with the flowering time. Large amount of rosette leaves indicates late flowering time. If there is a small amount of rosette leaves, flowering occurs earlier. The amount of rosette leaves is indicative of late or early flowering time in the same way as the amount of days from sowing to flowering. If flowering occurs late compared to wild type plant (vector control), transgenic, overexpressing lines have more time to produce rosette leaves until the first flower bud appears. Instead, the antisense line has less time to produce leaf mass until the flowering time. This means that the leaf mass of the antisense line is smaller than in wild type plant.

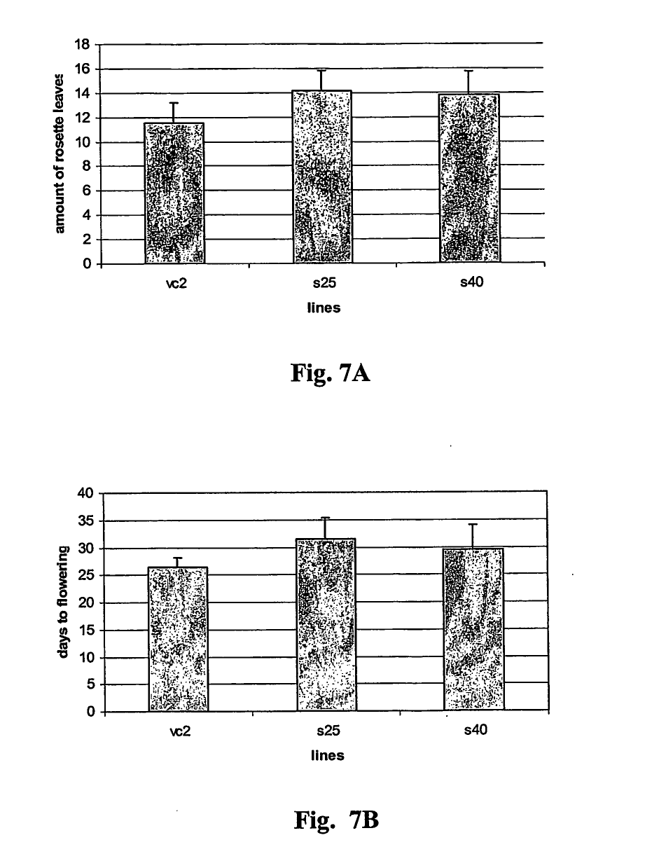

In SD and LD the amount of rosette leaves was higher in overexpressing lines than in vector controls (FIGS. 4A, 5A, 6A and 7A). In SD the amount of rosette leaves of antisense lines was smaller than in vector control lines (FIG. 4A). These results suggest that birch BpCol2 gene may act as a repressor of flowering in Arabidopsis.

Root lengths of BpCol2 transgenic Arabidopsis lines grown in long day (LD) conditions and in short day (SD) conditions were measured and photographed. The lengths of the roots of transgenic plant lines overexpressing BpCol2 gene were shorter than the length of the roots of vector control lines in SD and LD. Lines with antisense construct had longer roots compared to the vector control and sense lines in SD (Example 4). BpCol2 represses root growth in Arabidopsis.

It was noticed that the Arabidopsis cells are shorter in transgenic lines overexpressing the transgene. In addition the amount of cells decreased in overexpressing plants. In the root tip the of BpCol2 overexpressing line grown in long day (LD) conditions the cell morphology was altered compared to vector control including smaller cell size. In addition, the BpCol2 overexpressing line may have shortened elongation zone and alterations in meristematic zone including the lack of root cap compared to vector control (Example 10).

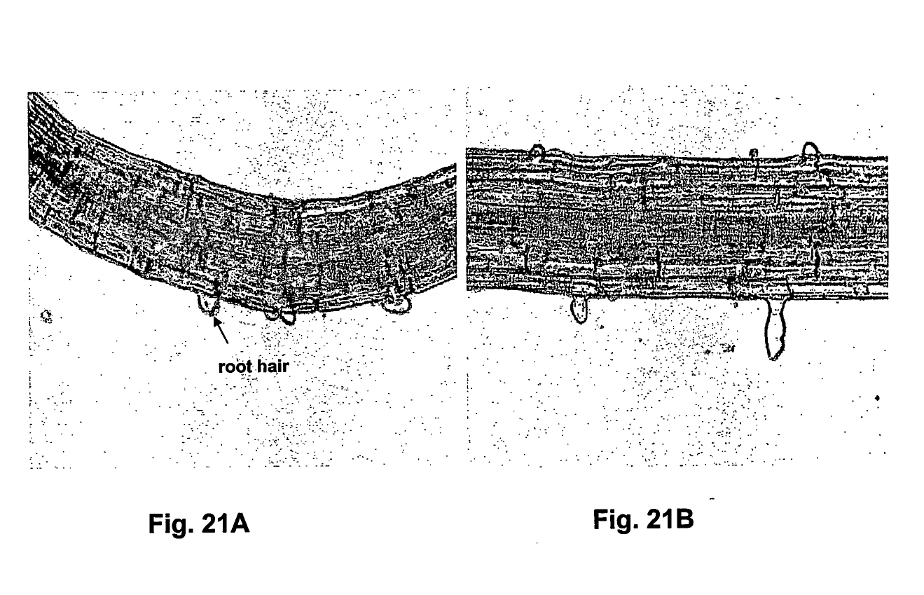

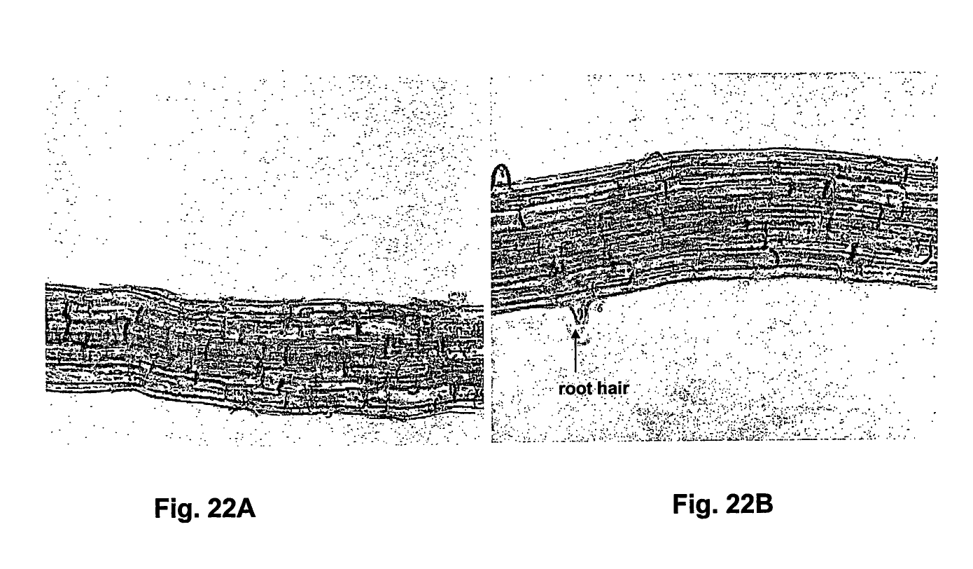

The changes in the cell structures of BpCol2 overexpressing line were more pronounced in short day (SD 10 h) than in long day (LD 16 h). The thin structure of the BpCol2 overexpressing line was clearer and the cells were shorter in SD compared to LD (FIGS. 21A and 22A). Especially BpCol2 overexpressing line may have more clearly shortened elongation zone in SD than in LD (19A and 20A). There might be alterations of BpCol2 overexpression line in meristematic zone including the lack of root cap compared to vector control in SD.

Root cell lengths of BpCol2 transgenic Arabidopsis plants (FIG. 21A) in differentiation zone in root tip were shorter compared to vector control (FIG. 21B) in long day conditions (LD) (16 h). Also in short day (SD) conditions the root cell length was shortened in transgenic Arabidopsis plants (FIGS. 22A and 22B).

T₂ generation transgenic Arabidopsis plants for Northern hybridization analysis were grown in short day (SD) (12 h light, 12 h dark) and in long day (LD) (16 h light, 8 h dark) conditions in growth rooms whereas T₃ generation plants were grown in greenhouse conditions with same photoperiods. Aerial parts of 4 plants were pooled for RNA extraction and Northern hybridization analysis was carried out with Dig-labeling method. Full length BpCol2 gene was a 1600 bp EcoRI-XhoI fragment cloned into pBluescript. 529 bp BpCol2 fragment was amplified using primers 5′-ctctgccgagtccataatcaa-3′ (SEQ ID NO:8) and 5′-taactgagtcggcacttggtt-3′ (SEQ ID NO:9) from the area between B-Box and CCT-domain (Example 9).

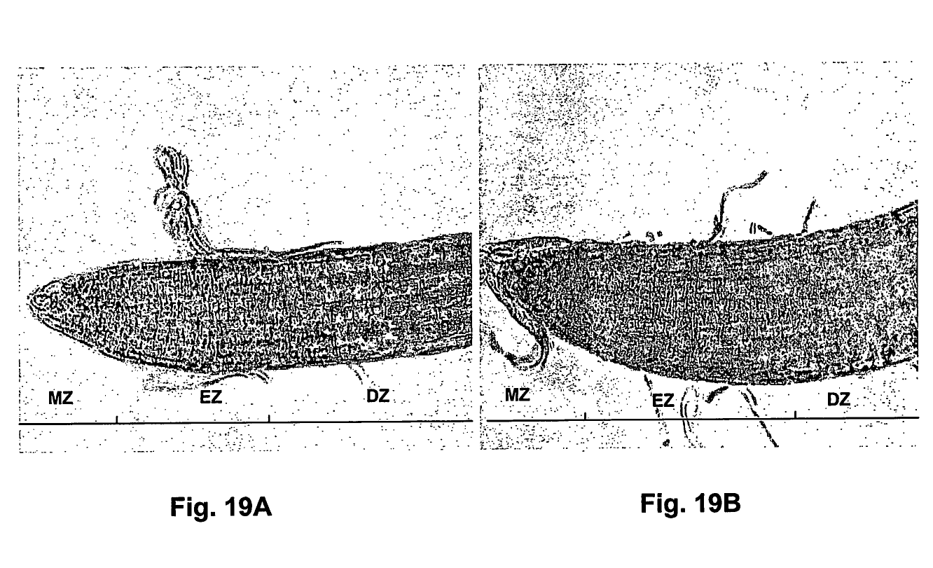

The roots of transgenic Arabidopsis plants were analyzed by microscope. The root tip of a transgenic Arabidopsis plants transformed with sense construct and grown in long day (LD) conditions is presented in FIG. 19A. The elongation region of the root tip is shorter than in the control plant presented in FIG. 19B.

The roots of overexpressing transgenic plants are smaller and the amount of cells is smaller in overexpressing sense lines than in control lines. There are possible changes in the root tips of overexpressing Arabidopsis plants. The cells are shorter in transgenic lines overexpressing the transgene. In addition the amount of cells has decreased in overexpressing plants.

Seeds from the T₃ generation BpCol2 overproducing Arabidopsis plants were sterilized and places on 1×MS media including 100 mg/L kanamycin at 4° C. for 2 days. After that plants were kept for 7 days under short day (SD) (10 h light, 14 h dark) or long day (LD) (16 h light, 6 h dark) in Sanyo growth chamber. After that roots were transferred onto the microscope slides and photographed with light microscope (20× magnification). Photographs were taken both from the root tips and from the differentiation zone of the roots. The cell morphology of BpCol2 overexpressing line root tip has altered compared to vector control including smaller cell size both in short and long day. In addition, the BpCol2 overexpressing line may have shortened elongation zone and alterations in meristematic zone including the lack of root cap compared to vector control. The changes of the cell structures of BpCol2 overexpressing line were more pronounced in short day than in long day. Especially BpCol2 overexpressing line had more clearly shortened elongation zone in SD than in LD. Root hair formation maybe reduced in BpCol2 overexpression line.

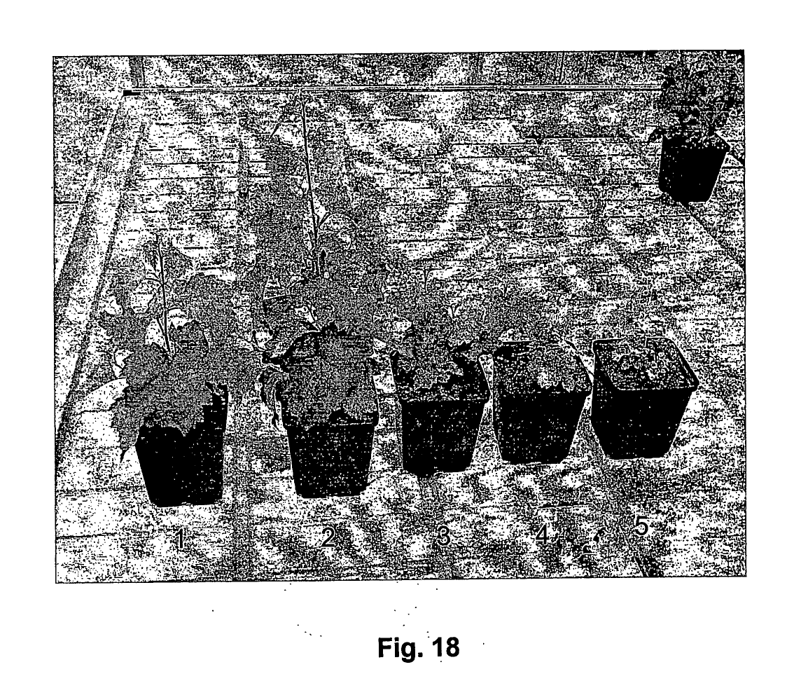

Transgenic birches carrying a BpCol2 transgene in antisense orientation grew faster than the wild type plant whereas overexpressing lines grew more slowly than the wild type plant in long day (LD) conditions. There were slight differences between the plant lines. The plant line 11 carrying a sense gene had the slowliest growing phenotype but the plant lines 15 and 20 with sense genes had also slowly growing phenotype (FIG. 18). This suggests that BpCol2 controls growth in birch.

Transgenic birch plants were grown in long day (LD) (23 h light, 1 h dark) conditions in a greenhouse. The height of the 2 months old plants was measured and photographed with digital camera In LD conditions the transgenic plants overexpressing BpCol2 gene were smaller than the wild type plants and the transgenic antisense plant line. In contrast, the growth of the antisense line was better than the growth of wild type.

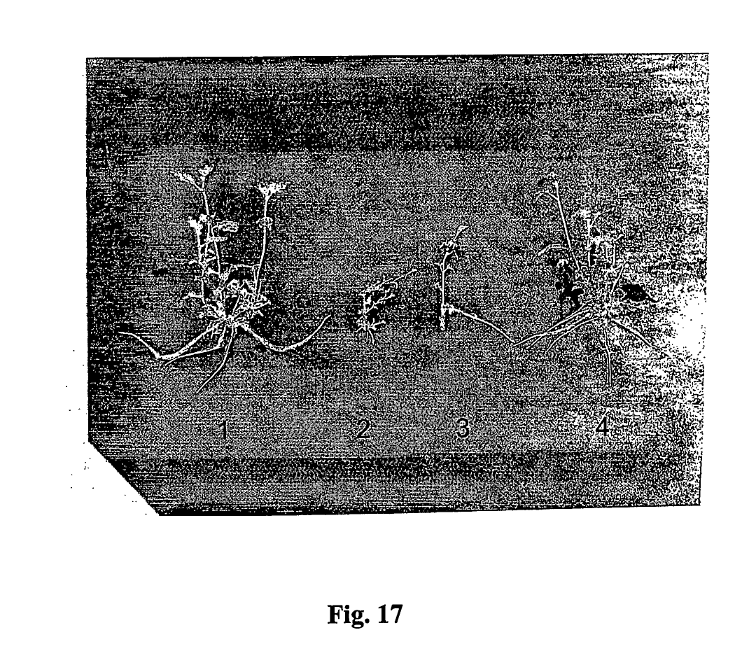

The transgenic birches containing an antisense construct had longer roots and overexpression lines had shorter roots compared to wild type plant in long day (LD) conditions. In contrast, the roots of the plants carrying a sense construct lines were shorter than the roots wild type plants. These results show that the overexpression of BpCol2 gene has a negative effect on the growth of birch whereas the antisense BpCol2 gene has a positive effect on the growth (FIG. 17).

The root lengths of the 4 weeks old BpCol2 transgenic birch plants grown in long day (LD) conditions were measured. The roots of the overexpression lines were shorter than in wild type plants. In contrast, antisense construct had a slight positive effect on root growth.

For the growth cessation experiment birch plants were transferred from long day (LD) to short day (SD) (12 h light/12 h dark) and the cessation of the growth in SD was followed regularly until the day 16. Total growth and days to growth cessation of BpCol2 transgenic birches were measured. There was 5 days difference in growth cessation in SD between these overexpressing lines s11 and s115 and vector control. BpCol2 overexpressing plant lines did not response to short day and were insensitive to 12 h photoperiod. There was not difference in growth cessation of BpCol2 overexpressing line 20 and wild type. However, the growth of the BpCol2 overexpressing line 20 was strong especially in the beginning of short day photoperiod compared to wild type. Thus this line is at least partially insensitive to short day photoperiod. The growth of the antisense line 5 ceased 2 days later in SD compared to WT. Total growth of BpCol2 overexpressing lines were higher than WT in SD (Example 12).

Branching of roots was studied in plants grown from seeds from T₃ generation BpCol2 overproducing Arabidopsis plants (Example 11). The presence of 35S::BpCol2 transgene in sense orientation may induce changes in apical dominance in BpCol2 overexpression lines grown in short day (SD) conditions (12 h light, 12 h dark). This caused strong formation of secondary roots and the weak growth of a primary root. In vector control the strong primary root and the smaller secondary roots were clearly seen. The root branching phenotype suggests changes in auxin hormone metabolism in BpCol2 overexpression plants.

The regulation of growth can be influenced using foreign promoters to alter the expression of the gene of the present invention. An inducible promoter can be used, for example a promoter induced by stress conditions. This is advantageous in that plant production, agriculture, horticulture and forestry and subsequent events such as seed set, may be timed to meet market demands, for example, in cut flowers or decorative flowering pot plants. Delaying flowering in pot to lengthen the period available for transport of the product from the producer to the point of sale and lengthening of the flowering period is an obvious advantage to the purchaser.

The nucleic acids of the present invention as well as the DNA constructs, transgenic cells and plants can be used in several applications in agriculture, forestry and horticulture. The spreading of plants through the roots can be controlled e.g. by preventing by preventing aggressive root formation. This can be done regulating the root growth by overexpressing BpCol2 or BpCol2 like genes in a plant. In horticulture plants with short roots may be desirable.

Another application of the present inventions is fighting against erosion or dense soil structure by enhanced root formation in agriculture, forestry and horticulture. Environmental factors affect expression of the transgene. Dry weather enhances rooting. Wet ground inhibits root formation.

Timing of flowering in female and male production in hybrid production can be influenced.

The growth of a plant can be inhibited to keep the plant in a particular size or stage of development for longer period of time, for example for producing plants of suitable height for a particular purpose.

The invention is described in more detail in the following examples in which the invention is applied to certain plants. These examples should not be interpreted to limit the scope of invention to said exemplified organisms. It is clear to one skilled in the art that the nucleic acid and amino acid sequences and a can be applied to in any plant.

### EXAMPLE 1

Identification of Birch (Betula pendula L. Roth) BpCol2 Gene

Two (2) cDNA libraries used for EST project had been generated from birch material grown in short day (SD) (12 h light, 12 h dark) conditions at low temperature (+4° C.). mRNA from birch cells was isolated and cDNA was synthesized from mRNA. cDNA was ligated to vector, lambda ZAP phagemid (Stratagene), which was transfected into E. coli bacterial cells as a phage particle. Sequences of birch ESTs were compared to the known sequences in the databases.

A birch homolog, BpCol2 (SEQ ID NO:1), to Arabidopsis CONSTANS gene was identified. A comparison of birch BpCol2 gene to the Arabidopsis CONSTANS gene is shown in FIG. 2. An alignment of an amino acid sequences encoded by BpCol2 and other birch BpCol homologs is presented in FIG. 3.

### EXAMPLE 2

Generation of Constructs Containing BpCol2 and Transformation of Arabidopsis Plants

The BpCol2 cDNA was ligated in sense and antisense orientations to the cauliflower mosaic virus (CaMV) 35S promoter (35S) and subcloned into the binary vector pDE1001 (Denecke, et al., EMBO J. 11:2345-2355, 1992) (FIGS. 1A and 1B). Binary vectors were mobilized into Agrobacterium tumefaciens C58C1 strain. Whole plants (T₀ generation) of Arabidopsis ecotype Col were transformed by dipping method (Clough et al., Plant Journal.16:735-743, 1998).

Homozygotes with a single active nptII locus were selected based on the segregation of kanamycin resistance in the T₂ generation. T₃ generation plants homozygous for the BpCol2 overproducing construct were also used for flowering time, root growth and expression analysis.

Transgenic Arabidopsis thaliana lines S25 and S40 contained the sense 35S::BpCol2 construct and the lines AS17 and AS18 contained the antisense 35S::BpCol2 transgene. Line VC2 contained the vector control construct 35S::leader sequence.

### EXAMPLE 3

Growth Conditions and the Measurement of Flowering Time in Arabidopsis

Arabidopsis seeds were sterilized and placed on 1 xMS (Murashige and Skoog) media including 100 mg/L kanamycin at +4° C. for 2 days. After that plants were kept for 7 days under short day (SD) (12 h light, 12 h dark) or long day (LD) (16 h light, 8 h dark) conditions in in vitro culture rooms. For the flowering time experiment 7 days old kanamycin resistant plants were transferred into the soil. Flowering time was measured under defined conditions by growing plants in short day (SD) and long day (LD) conditions in controlled growth rooms or greenhouses at +22° C. A short day comprised a photoperiod of 12 hours with 36 Watt lamps. This provided a level of 100 .mu.moles photons m⁻²s⁻¹. A long day (LD) photoperiod was 16 hours light with 100 .mu.moles m⁻²s⁻¹.

Flowering time was measured by counting the number of rosette leaves (FIGS. 4A, 5A, 6A and 7A) and days (FIGS. 4B, 5B, 6B, and 7B) from sowing at the time point when the first flower bud was visible. The amount of rosette leaves correlates with the flowering time. Large amount of rosette leaves indicates late flowering time. If there is a small amount of rosette leaves, flowering occurs earlier. The amount of rosette leaves is indicative of late or early flowering time in the same way as the amount of days from sowing to flowering. In SD and LD conditions the amount of rosette leaves was higher in overexpressing plant lines than in plants containing a vector control (FIGS. 4A, 5A, 6A and 7A). In SD conditions the amount of rosette leaves of antisense lines of T₂ generation plants were smaller than in a vector control line (FIG. 4A). In LD conditions antisense did not have any effect on flowering (FIG. 5A).

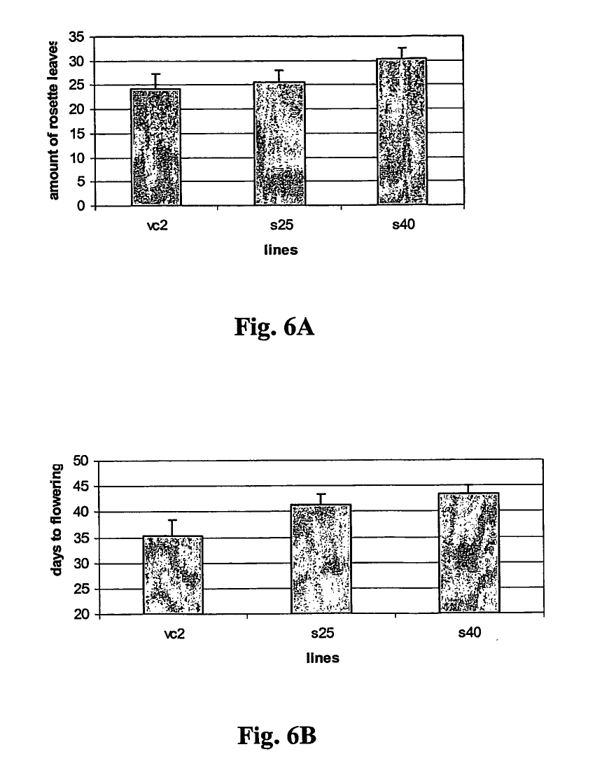

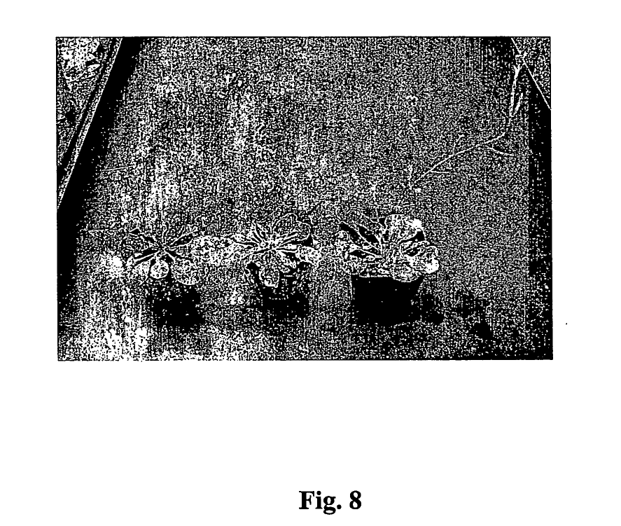

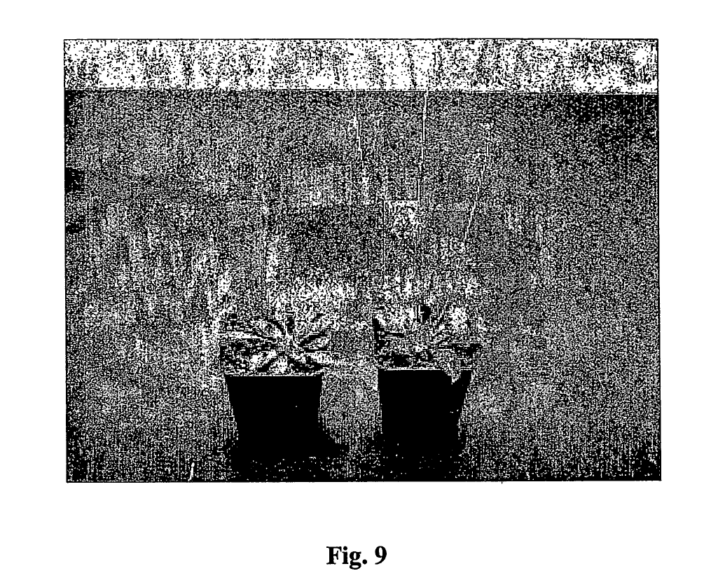

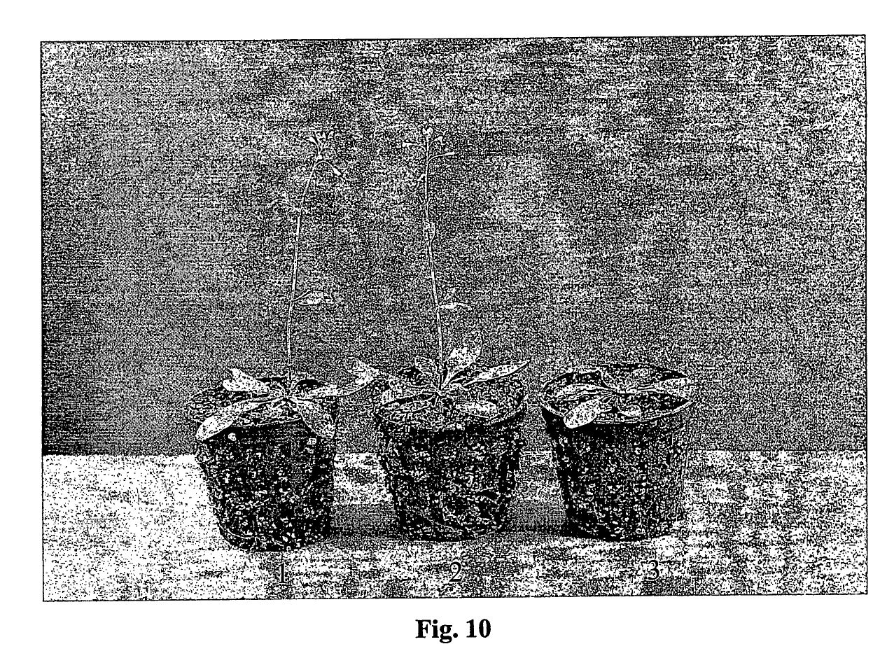

The amount of days to flowering in BpCol2 overexpressing lines was higher than with vector control lines in SD and LD conditions which indicate the late flowering of BpCol2 overexpressing lines (FIGS. 4B and 6B). The two plant lines with an antisense construct flowered earlier than the vector control in SD conditions (FIG. 4B). In LD there was no difference in flowering time between antisense and vector control line (FIG. 5B). Data was presented as mean±t.SE (p 0.05). The phenotype pictures from T₂ and T₃ generation plants of BpCol2 overexpressing transgenic lines showed the late flowering phenotype both in SD and LD conditions (FIGS. 8, 9 and 10). The early flowering phenotype of antisense construct was clear in T₂ generation plants and only in SD conditions (FIG. 8).

### EXAMPLE 4

Growth Conditions and the Measurement of the Length of the Root in Arabidopsis

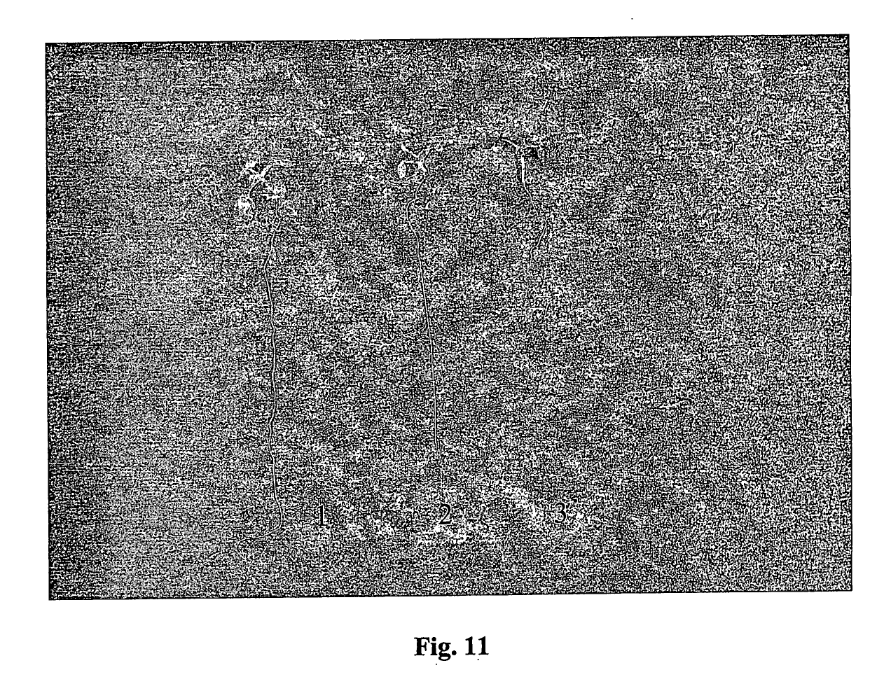

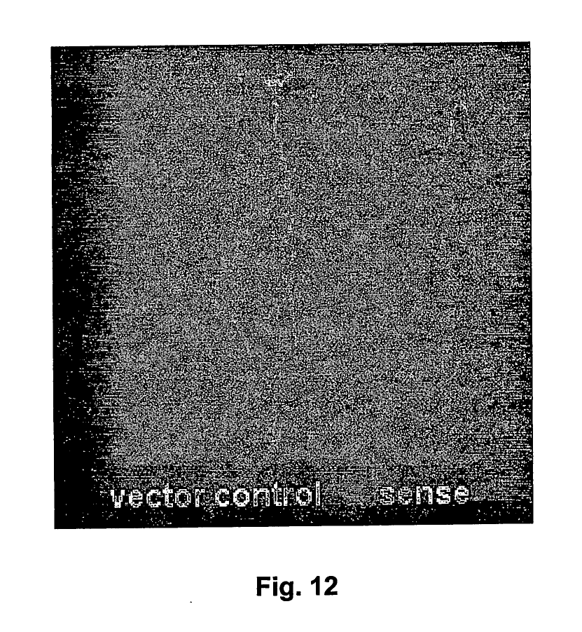

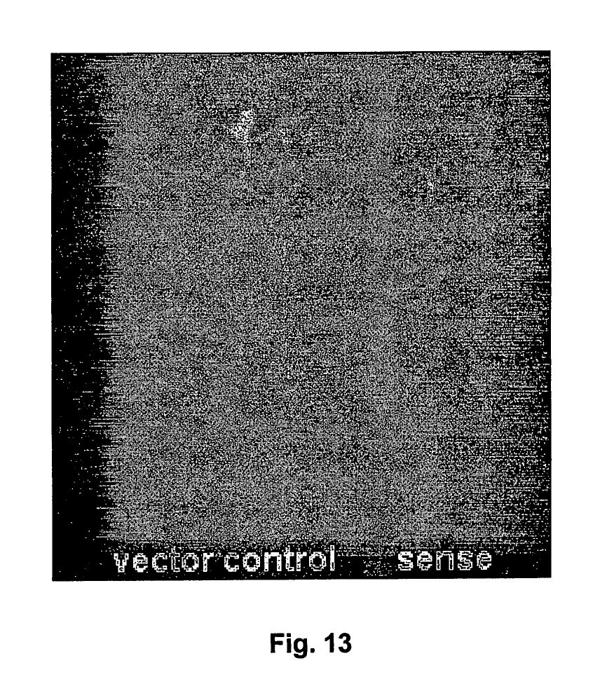

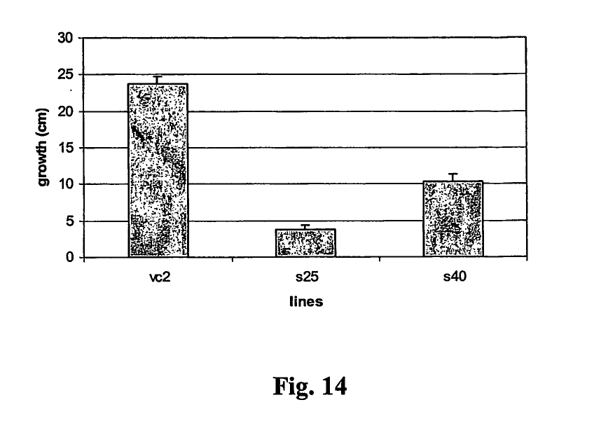

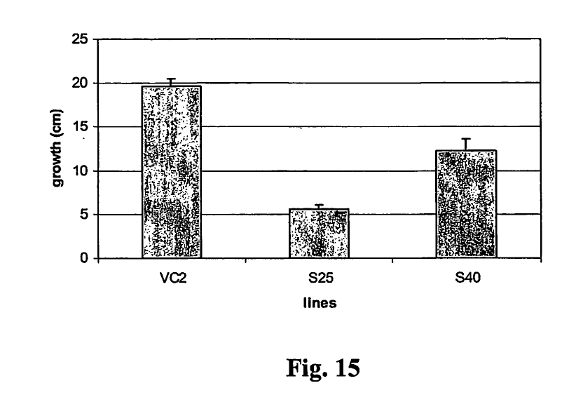

Arabidopsis seeds were sterilized and placed on 1×MS (Murashige and Skoog) media including 100 mg/L kanamycin at +4° C. for 2 days. After that plants were kept for 7 days under short day (SD) (10 h light, 12 h dark) or long day (LD) (16 h light, 8 h dark) conditions in in vitro culture rooms. 7 days old roots of BpCol2 transgenic Arabidopsis roots were measured and photographed. Root lengths were measured with a transparent ruler held adjacent to plants growing on vertically oriented petri dish. In T₂ and T₃ generation plants the lengths of the roots of transgenic plant lines overexpressing BpCol2 gene were shorter than the length of the roots of vector control lines in SD and LD conditions (FIGS. 11, 12, 13, 14 and 15). The Arabidopsis lines carrying an antisense construct had longer roots compared to the vector control and sense lines in T₂ generation (FIG. 11).

### EXAMPLE 5

Generation of BpCol2 Constructs and Transformation of Birch Plants

The BpCol2 cDNA was ligated in sense and antisense orientations to the cauliflower mosaic virus (CaMV) 35S promoter (35S) and subcloned into the binary vector pDE1001 (FIG. 1A and 1B) (Denecke et al., EMBO J. 11:2345-2355, 1992). Binary vectors were mobilized into Agrobacterium tumefaciens C58C1 strain. The leaves and shoots of birch ecotype JR 1/4 were transformed using a birch transformation protocol (Keinonen-Mettälä et al., Plant Cell Reports. 17:356-361, 1998). Homozygotes with a single active nptII locus were selected based on the segregation of kanamycin resistance. Kanamycin resistant plants were transferred into the soil.

Transgenic birch lines S11, S15 and S20 contained the sense 35S::BPCOL2 construct and the line AS 5 contained the antisense 35S::BPCOL2 transgene. A wild type birch was used as a control.

### EXAMPLE 6

Growth Conditions and Measurement of the Growth of Transgenic Birches

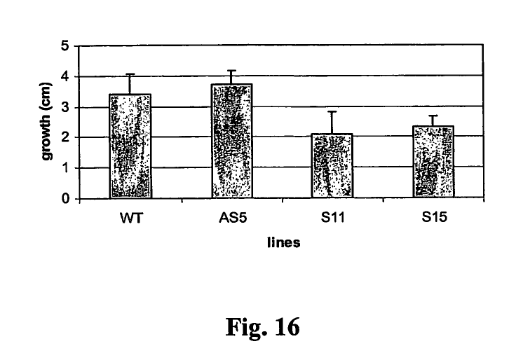

Plants were propagated by in vitro shoot culture in WPM-media including 1 mg/L BAP and grown in controlled growth chamber at 19° C. under photoperiod of 23 h light and 1 dark for 6 weeks. Photon flux density of the light in chamber was 120 μmol⁻² s⁻¹. After that plants were transferred into the WPM-media including 1 mg/L IAA in which plants were grown for 4 weeks. For root length measurement 4 plants/line were laid on the wet Whatman membrane and measured with transparent ruler (FIGS. 16 and 17). Data are presented as mean±t.SE (p 0.05). After 10 weeks plants were transferred into the soil which comprised 1:1 (v/v) vermiculite:soil mixture and grown in LD (23 h light, 1 h dark) conditions in a greenhouse with light level of 300 μmol⁻² s⁻¹. The height of the 2 months old plants was measured and photographed with digital camera (FIG. 18).

In LD conditions the transgenic plants overexpressing BpCol2 gene were smaller than the wild type plants and the transgenic antisense plant line. In contrast, the growth of the antisense line was better than the growth of wild type (FIG. 18). The roots of the antisense plants were longer than the roots of the wild type plants. In contrast, the roots of the plants carrying a sense construct lines were shorter than the roots wild type plants (FIGS. 16 and 17). These results show that the overexpression of BpCol2 gene has a negative effect on the growth of birch whereas the antisense BpCol2 gene has a positive effect on the growth.

### EXAMPLE 7

Birch Transformation Protocol

5 weeks old birch explants grown in MS+1 mg/l BAP were used ready for transformation. Leaves of birch plants were cut in water+750 mg/l ascorbic acid solution. Ascorbic acid was measured to 10 ml of water and sterilized to the 1 liter of water. Leaves were transferred from the ascorbic acid solution to the MSG-media including 1 mg/l BAP and 2 mg/l 2,4-D hormones. These media were kept for 5 days at room temperature in dark. Agrobacterium was grown in 3 ml liquid LB-cultures including antibiotics (claforan 100 mg/L, carbencillin 100 mg/L and spectinomycine 100 mg/L) at 30° C. over night. 300 μl from each Agrobacterium cultures was transferred into a new 10 ml LB-liquid and grown for 3 hours at 30° C. Agrobacterium-solution was centrifuged 4000 rpm for 5 min. Pellet was resuspended in 10 ml of MSG-gene transfer solution without PVPP and acetosyringone.

The edges of the birch leaves were cut and small cuts were made on the surface of the plant tissue with a sharp knife. The explants which were dried on a sterile, autoclaved paper towel were moved into the Agrobacterium solution (made above). The explants were shaken in the Agrobacterium solution for approximately 30 seconds. The explants were removed from agrosuspension onto the MSG-medium. Media were incubated for 2 hours at room temperature in dark. The explants were transferred from MSG-media into the gene transfer solution including 25 ml MSG+MES 25 mM, 1% PVPP and acetosyringone 20 mg/l and kept at room temperature in dark, shaking slowly 75-100 rpm. The gene transfer solution was changed every day in the morning and late afternoon. Also fresh and sterile acetosyringone was added in the morning and late afternoon. The explants were kept in MSG medium for 3 fill days.

The explants were rinsed with 3×1:10 MS-solution and transferred after rinsing into the agro-killing solution which included 25 ml MS-solution+1 mg/i BAP+2 mg/l NAA+1% PVPP, MES 25 mM, DTT 0.10 mg/ml, claforan 500 mg/l and vancomycin 500 mg/l. The explants were incubated in agro-killing solution in dark and cold room (+4° C.) with shaking slowly 75-100 rpm for one week and the solution was changed every day.

The explants were rinsed with MS-solution and dried after rinsing with autoclaved paper towel. Dried explants were transferred onto the solid growth media including WPM+2 mg/l TDZ, claforan 200 mg/l, ticarcillin 200 mg/l, PVP 200 mg/l, Ag-thiosulphate 7.5 μM, Kanamycin 75 mg/l and Gelrite 3.25 g/l. The explants were kept for 4 week at growth room.

After 4 weeks the explants were transferred onto the new media containing WPM+2 mg/l TDZ, ticarcillin 200 mg/l, PVP 200 mg/l, Ag-thiosulphate 7.5 μM, Gelrite 3.25 g/l and Kanamycin 75 mg/l. The explants were kept for 4 week at growth room After 4 weeks the explants were transferred onto the new media containing WPM+TDZ 2 mg/l, ticarcillin 100 mg/l, Ag-thiosulphate 7.5 μM, Gelrite 3.25 g/l. The explants were kept for 4 weeks at growth room.

After 4 weeks green “tissue balls” were transferred onto the new media containing WPM+1 mg/l BAP, Kanamycin 100 mg/l, vancomycin 50 mg/l and Gelrite 3.25 mg/l

### EXAMPLE 8

Analysis of Transgenic Lines

BpCol2 might be involved in SD or LD photoperiod pathway. Thus, expression analysis of Arabidopsis and birch BpCol2 transgenic plants grown in both SD and LD conditions is done using Northern analysis. Expression of selected genes can thus be also studied using a different method e.g. real-time PCR.

Affymetrix Chip analysis of transgenic Arabidopsis plant lines are done to detect the target genes of birch BpCol2 gene in LD and SD photoperiod pathway. DNA micro array analysis using the birch chip is performed. The first experiments using both sense and anti-sense transgenic plants are done with a stress-specific chip and later with a full-size chip.

Also microscopic analysis of birch cell morphology and analysis of wood structure are to be done.

Red- and far-red light experiments of transgenic BpCol2 Arabidopsis plant lines are done to see if there is a response to these light conditions. Furthermore this experiment might give information under which sensory pathway BpCol2 is e.g. phytochromes, cryptochromes.

### EXAMPLE 9

Northern Analysis of Transgenic Arabidopsis Plants

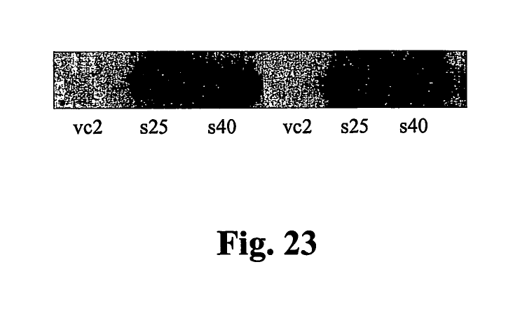

T₂ generation transgenic Arabidopsis plants for Northern hybridization analysis were grown in short day (SD) (12 h light, 12 h dark) and in long day (LD) (16 h light, 8 h dark) conditions in growth rooms whereas T₃ generation plants were grown in greenhouse conditions with same photoperiods. Aerial parts of 4 plants were pooled for RNA extraction and Northern hybridization analysis was carried out with Dig-labeling method (Roche). Full length BpCol2 gene was a 1600 bp EcoRI-XhoI fragment cloned into pBluescript (Stratagene). 529 bp BpCol2 fragment was amplified using primers 5′-ctctgccgagtccataatcaa-3′ (SEQ ID NO:8) and 5′-taactgagtcggcacttggtt-3′ (SEQ ID NO:9) from the area between B-Box and CCT-domain. In T₂ and T₃ generation plants the expression level of BpCol2 gene of overexpressing lines was high both in short and long day (FIG. 23)

### EXAMPLE 10

Microscopic Analysis of Arabidopsis Roots

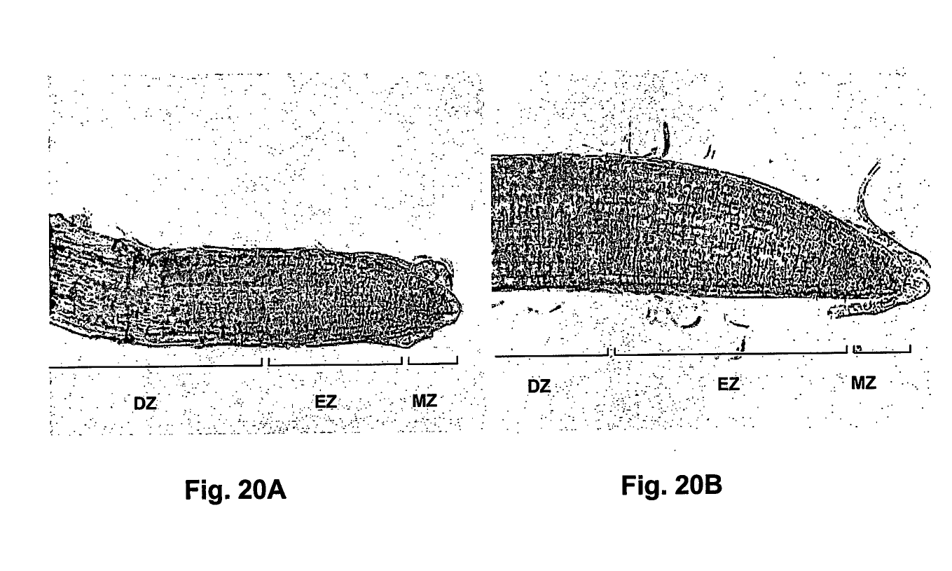

Seeds from the T₃ generation Arabidopsis plants were sterilized and places on 1×MS media including 100 mg/L kanamycin at 4° C. for 2 days. After that plants were kept for 7 days under short day (SD) (10 h light, 14 h dark) or long day (LD) (16 h light, 6 h dark) in Sanyo growth chamber. After that roots were transferred onto the microscope slides and photographed with light microscope (20×magnification). The roots of transgenic Arabidopsis plants were analyzed by microscope (ZEISS Axioplan 2 imaging). Photographs were taken both from the root tips and from the differentiation zone of the roots (FIGS. 19-22). The cell morphology of BpCol2 overexpressing line root tip has altered compared to vector control including smaller cell size both in short and long day. In addition, the BpCol2 overexpressing line may have shortened elongation zone and alterations in meristematic zone including the lack of root cap compared to vector control. The changes of the cell structures of BpCol2 overexpressing line were more pronounced in short day than in long day. Especially BpCol2 overexpressing line had more clearly shortened elongation zone in SD than in LD. Root hair formation maybe reduced in BpCol2 overexpression lines.

### EXAMPLE 11

Branching of Roots

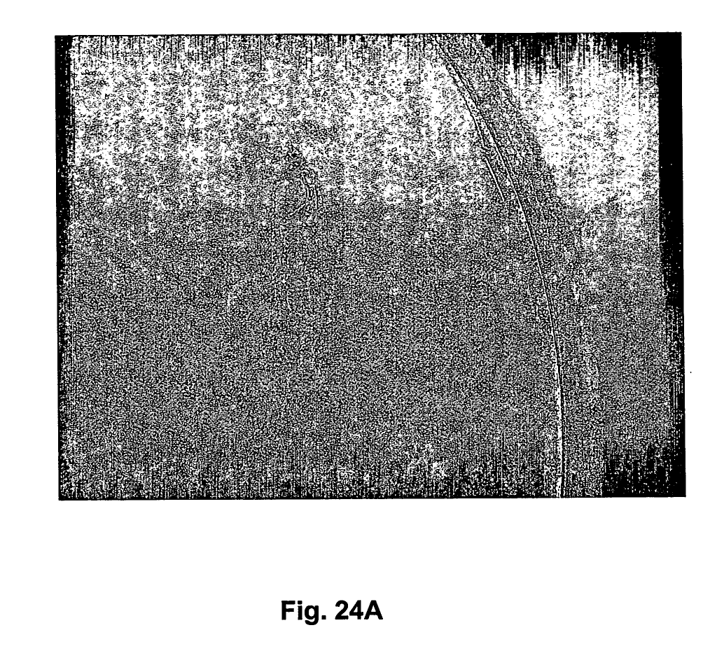

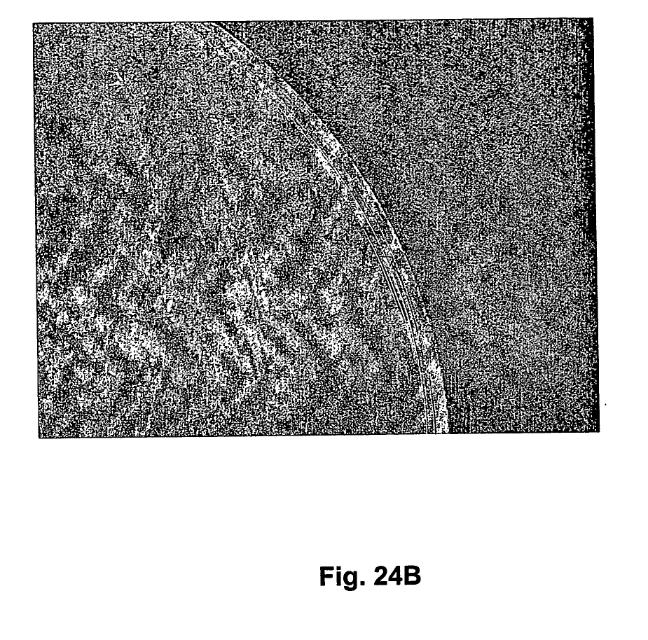

Seeds from T₃ generation Arabidopsis plants were sterilized and placed on 1×MS media including 100 mg/L kanamycin at 4° C. for 2 days. After that plants were kept for 7 days under short day (SD) (12 h light, 12 h dark). After one week plants were transferred onto the 1×MS media and kept 7 days under short day. Photograph was taken with digital camera. 35S::BpCol2 transgene in sense orientated plants induced the strong formation of secondary roots and the weak growth of primary root (FIG. 24A). In vector control the strong primary root and the smaller secondary roots were clearly seen (FIG. 24B). The root branching phenotype suggests changes in auxin hormone metabolism in BpCol2 overexpression plants.

### EXAMPLE 12

Cessation of Growth in Transgenic Birch Plants

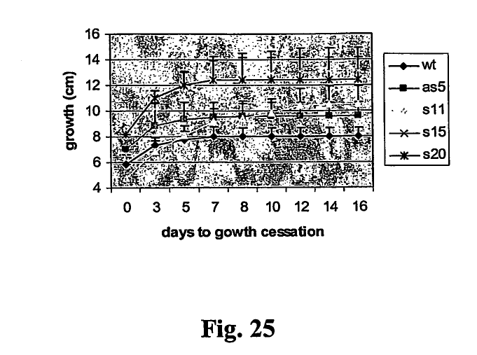

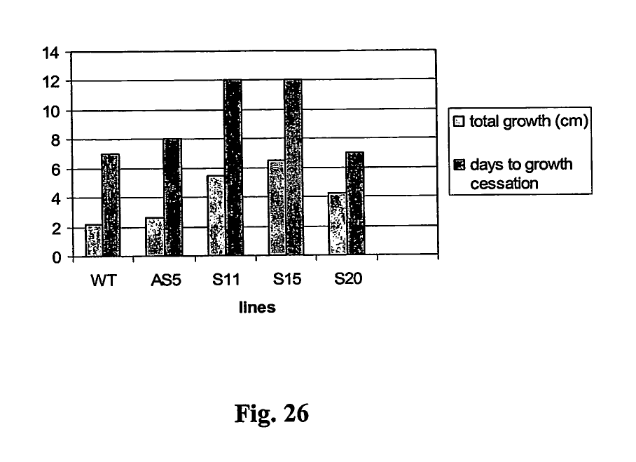

Plants were propagated by in vitro shoot culture in WPM-medias including 1 mg/L BAP and grown in a controlled chamber at 19° C. under photoperiod of 23.0 h for 6 weeks. Photon flux density of the light in chamber was 120 μmol⁻² s⁻¹. After that plants were transferred into the WPM-medias including 1 mg/L IAA in which plants were grown for 4 weeks. After 10 weeks plants were transferred into the soil comprised a 1:1 (v/v) vermiculite:soil mixture and grown in LD (23 h light, 1 h dark) three months with light level of 300 μmol⁻² s⁻¹ in a greenhouse. For the growth cessation experiment plants were transferred from LD to the SD (12 h light/12 h dark) and the timing of the cessation of the growth in SD was followed regularly during days 0, 3, 5, 7, 8, 10, 12, 14 and 16. The stem elongation of the BpCol2 overexpressing lines 11 and 15 did not stop within 7 days as did the wild type. BpCol2 overexpressing lines 11 and 15 continued growth in SD until the day 12. There was 5 days difference in growth cessation in SD between these overexpressing lines and vector control (FIGS. 25 and 26). 35S::BpCol2 in sense orientated plants did not response to short day and was insensitive to 12 h photoperiod. There was not difference in growth cessation of BpCol2 overexpressing line 20 and wild type. However, the growth of the BpCol2 overexpressing line 20 was strong especially in the beginning of short day photoperiod compared to wild type (FIG. 25). Thus this line is at least partially insensitive to short day photoperiod. The growth of the antisense line 5 ceased 2 days later in SD compared to WT. Total growth of BpCol2 overexpressing lines were higher than WT in SD (FIG. 26).

1-15. (canceled)

16. A method of influencing the growth of plant roots, said method comprises the steps of
(i) introducing at least one nucleic acid molecule according to claim 27 into a plant cell, optionally such that expression of the nucleic acid molecule is under the control of a promoter, and over-expressing the nucleic acid molecule and
(ii) regenerating a plant from said transformed plant cell, wherein the polypeptide encoded by the introduced nucleic acid molecule is expressed in the transformed plant.

17. The method according to claim 16, wherein the influencing of plant roots results in decreasing the growth of plant roots.

18. A method of influencing the growth of plant roots, said method comprising the steps of
(i) introducing at least one nucleic acid molecule or a fragment thereof according to claim 27 in antisense orientation into a plant cell, optionally such that expression of the nucleic acid molecule is under the control of a promoter and
(ii) regenerating a plant from said transformed plant cell, wherein the polypeptide encoded by the introduced nucleic acid molecule is expressed in the transformed plant.

19. The method according to claim 18, wherein the influencing of plant roots results in promoting the growth of plant roots.

20. A method of influencing growth in a plant, said method comprises the steps of
(i) stably incorporating into a plant genome a DNA construct of claim 29 comprising a promoter and a nucleic acid coding sequence encoding gene capable of modifying the growth or length of the plant or growth or length of roots and
(ii) regenerating a plant having an altered genome.

21-26. (canceled)

27. An isolated nucleic acid molecule encoding a polypeptide selected from the group consisting of SEQ ID NO:2, SEQ ID NO:3, SEQ ID NO:4, SEQ ID NO:5, SEQ ID NO:6 and SEQ ID NO:7 or fragments thereof capable of hybridizing to said nucleic acids or a complement thereof.

28. An isolated nucleic acid molecule or a complement thereof capable of hybridizing to a nucleic acid sequence selected from the group of SEQ ID NO: 1, a nucleic acid sequence encoding a polypeptide SEQ ID NO:2, SEQ ID NO:3, SEQ ID NO:4, SEQ ID NO:5, SEQ ID NO:6 and SEQ ID NO:7.

29. The isolated nucleic acid of claim 27, wherein the nucleic acid sequence further comprises at least one regulatory sequence for expressing said nucleic acid.

30. A vector encoding at least one nucleic acid according to claim 27.

31. A host cell encoding a nucleic acid according to claim 27.

32. A plant transformed with a nucleic acid according to claim 27.

33. A plant seed of claim 32.
